(19) United States
(12) Patent Application Publication
(10) Pub. No.: US 2025/0296086 A1
(43) Pub. Date: Sep. 25, 2025

(54) BLOOD ANALYSIS SYSTEM

(71) Applicant: Bal Chander, Kangra (IN)

(72) Inventor: Bal Chander, Kangra (IN)

(21) Appl. No.: 19/230,390

(22) Filed: Jun. 6, 2025

Related U.S. Application Data
(63) Continuation-in-part of application No. 17/609,281, filed on Nov. 5, 2021, now Pat. No. 12,350,039.

(51) Int. Cl. B01L 3/00; G01N 1/40

(52) U.S. Cl. CPC B01L 3/502753; G01N 1/4077; B01L 2300/0627; B01L 2300/0681; B01L 2300/0832; B01L 2400/0409; B01L 2400/043

(57) ABSTRACT

A blood analysis system comprises: a centrifugation unit to receive blood of a subject to hold first capturing molecules, wherein one first capturing molecule binds with, to chemically capture, one molecule or ion that causes coagulation of the plasma and centrifuge to suspend cellular components with a minimal plasma along with a number of the first capturing molecules; a detection unit to receive the plasma with remaining number of the first capturing molecules and unbound molecules and ions, but without the cellular components, to determine concentration of one or more types of molecules and/or ions; and a correction unit to correct the determined concentration of the one or more types of molecules and/or ions.

The present application is for a continuation-in-part application to the U.S. application Ser. No. 17/609,281, and the subject matter of present application comprises an improvement in, or a modification of the subject matter claimed in the specification of the U.S. application Ser. No. 17/609,281.

### TECHNICAL FIELD

The subject matter described herein, in general, relates to physiological and pathological system, and in particular relates to blood analysis and correction system.

### BACKGROUND

Body parameters associated with heart and brain activities, and oxygen saturation in mammals can be continuously monitored in real time via ECG machines, EEG machines, BP machines, pulse oxymetry systems, etc. These instruments yield real time information about functionality of vital organs like heart, brain and lungs. However, the underlying molecular/ionic parameters in the blood that have direct bearing on the functions of different organs are not monitored in real time and therefore, we do not know how fluctuations of the blood parameters affect different organs. This assumes importance in critically ill patients. A few modalities are available indeed for instance ion-selective electrodes and implanted devices which can give the reading of only a few molecules in real time (Ref.1 and Ref.2 enlisted at the end of the specification).

### BRIEF DESCRIPTION OF THE DRAWINGS

The following detailed description references the drawings, wherein:

FIG. 1 illustrates an example of a blood analysis system, in accordance with the present subject matter;

FIG. 2 illustrates an example of an ion separation unit, in accordance with the present subject matter;

FIG. 3 illustrates an example of a correction unit, in accordance with the present subject matter;

FIG. 4 illustrates an example of a filtration unit, in accordance with the present subject matter;

FIG. 5 illustrates an example of a concentration unit, in accordance with the present subject matter;

FIG. 6A to 6D illustrate examples of a detection unit, in accordance with the present subject matter; and

FIG. 7 illustrates an example of a blood analysis system, in accordance with the present subject matter.

### DETAILED DESCRIPTION

At present, if continuous monitoring of all the hormones in the blood is required, one would have to take repeated blood samples and wait for the results to come which, even if a dedicated machine is available, may take at least an hour. Therefore, the patient not only loses blood but the values obtained of the said parameter(s) is/are not in real-time and/or continuous. We end up with values obtained at intervals oblivious to the fact that even hourly measurement does not necessarily mean there must have been no fluctuation of the values during the hour or any interval for that matter. It can be stated that any morbid state or even physiological variations are reflected in blood in the form of variation of levels of molecular and/or ionic entities and that is the reason why measurements of blood parameters make the cornerstone of patient management.

The principle underlying most of the blood, including plasma or serum tests is selective reaction between a molecule and ionic entity and externally added chemical reagents that results in a reaction product. The reaction product can be measured with the help of some kind of spectroscopy and quantified. The quantity of reaction product is related to the molecule/ionic entity under investigation.

Given, the fact that there are thousands of molecules in the blood, selective chemical reaction is a must without any erroneous reaction which might confound the results. However, there are means that can be employed to measure blood parameters without the use of chemical reactions and the foremost among them is Pulse oxymetry. Pulse oxymetry uses the principal of Absorbance. Oxygenated and deoxygenated hemoglobins absorb different lights of wavelength 660 (red) and 940 (infrared). The ratio of red and infrared absorbance is proportional to the oxygenated haemoglobin (blood). It is a non invasive real time measurement of blood oxygen (Ref.3).

There are other techniques for continuous in vivo real time measurement of molecules like dopamine (Ref.4), serotonin (Ref.5), glutamate (Ref.6) and lactate (Ref.7) which rely on chemical reaction and therefore have the limitation of measuring only a few entities. Surface based biosensors surface plasmon resonance (SPR) (Ref.8), quartz crystal microbalances (QCM), field-effect transistors (FET), and microcantilevers have limited practical application when exposed to whole blood since the latter has very high molecular load which leads to non specific adsorption (Ref.9). Therefore, surface biosensors too have limitation of number of molecules that can be measured in real time. Novel approaches like electrochemical aptamer-based (E-AB) sensors that utilizes conformational changes in response to specific molecules in whole blood to measure the concentration also has the limitation since each aptamer needs to be synthesised for detection of different molecules (Ref.10).

If one were to use surface based biosensors for the whole blood without unwanted non-specific adsorption, the solution would be to reduce the number of type of molecules in the blood as much as possible so that the chances of non-specific adsorption are reduced too. The reduction in the type of molecules can be achieved by making use of semi-permeable membranes (one or more than one) with different properties so that only the molecule of interest is the final product which then can be measured utilizing sensors based on surface plasmon resonance (SPR), quartz crystal microbalances (QCM), field-effect transistors (FET), microcantilevers or E-AB with markedly reduced probabilities of nonspecific reactions or interactions.

Alternatively, the product of filtration (a few types of molecules) can be measured using any one or combination of different types of spectroscopies or biosensors.

The separation or filtration of types of molecules cannot be achieved in vivo except when only a few types of molecules have to be measured, therefore much more pragmatic solution for measuring more types of molecules in real time would be to carry out the separation or filtration in an extracorporeal system.

Added advantage of extracorporeal system is the controlled correction of level of any or more than one molecule that can be carried out using a novel method of chemical capture without use of drugs and therefore adverse effects. The principle is similar to employed in specific type of real time PCR where mRNA is separated from reaction mixture using oligodeoxythymidylate [oligo (dT) 25]-coated magnetic particles (Ref.11).

There are existing medical equipment used for blood purification based on use of semi-permeable membrane with or without adsorption columns.

The separation or filtration of molecules is routinely carried out in Dialysis where a few types of molecules are filtered out by a semi-permeable membrane. In order to make the filtration as specific as possible and to preserve the ionic balance, the dialysing fluid is constituted such that there is minimal or no disturbance of blood pH or electrolyte balance. The filtered molecules are discarded along with the dialysing fluid.

The key differences between dialysis and the system described in the present disclosure is that:
i) In latter instead of dialysing fluid, pure water is used.
ii) The target entities are measured in real time preferably using non chemical methods.
iii) Only the target filtered product is captured and discarded along with the pure water.
iv) Rest of the (known and unknown) molecules and ions are retained in the System.

The purpose of sequential filtration in the system described herein is primarily reduction of types of molecules either in filtrate or retentate which in turn makes it easier to:
i) detect and quantify a given molecule or molecules with more precision.
ii) design capturing molecules with reduced probability of non-specific reactions.

Blood is composed of cellular (red and white blood cells) and acellular (molecular and ionic) components, hereinafter referred to as detectables. If one were to measure all the molecules (excluding ions for the time being) using conventional means, it would take unacceptable amount of blood from the patient considering the fact that there are approximately 30,000 native molecules at any point of time circulating in the blood not to mention drugs with metabolites and microorganism toxins in different clinical situations.

To measure the concentration of detectables in real-time over hours/days or even longer periods of time would be invaluable to not only understand the normal mammalian including human physiology but more importantly changes in different morbid states. Such understanding is vital in order to completely understand pre-mortem changes and the only way to understand the dynamics between thousands of entities present in the blood and indirectly between tissue/organs.

The subject matter described herein relates to blood analysis systems for analysis of blood for various purposes including, but not limited to, medical diagnostics and patient management. The blood analysis system is designed with the view of gaining complete understanding of the molecular basis of physiological and pathological states of mammals as reflected in blood and the use of the attained knowledge to treat disease conditions and/or promote health. The blood analysis systems of the present subject matter are designed for real-time and continuous assessment and manipulation of the levels of all molecules and ionic entities in blood. The blood analysis system of the present subject matter may be referred to as an extracorporeal system, capable of accurate measurement of all molecules and ionic entities without the use of any chemical reaction.

The blood analysis system of the present subject matter is designed to reduce highly complex mixture of molecules in blood to multiple channels each ideally containing only one type of molecule via sequential combination of filtration units with first one filtering out everything but the cells and second one filtering out all molecules except the molecules with parameters within defined range and so on till the last unit which ideally carries only single type of molecule. The blood analysis system of the present subject matter can be used to evaluate all the entities in blood however it can also be contracted to target select group of molecules or just ionic entities.

The blood analysis system of the present subject matter is capable of correction (addition or extraction) of any molecular/ionic level in blood with the use of highly selective chemical reactions before returning the blood back into the body. Since selective chemical reaction will occur in the blood analysis system and blood would be returned to the body without any undesirable alterations, the adverse effects of treatment/intervention are minimal or none in comparison to the conventional methods of diagnostics and treatment for many diseases.

The isolation of individual molecular/ionic entities or their separation into small groups in the blood analysis system is done with the sequential use of semi-permeable membranes with or without electromagnetic forces and with or without centrifugation. The semi-permeable membranes separate the given entities based on their molecular cut-off size while the electromagnetic forces separate them based on their charge/dipole moment. At every level of separation there are concentration and detection units which make use of electromagnetic waves or electromagnetic fields or voltage difference possibly but not exclusively the form of selective electrodes for detecting and measuring the levels of entities.

Extraction of undesired molecules/ions from a mixture of molecules/ions, when required, is done with the use of specific chemical reaction of the molecule/ion of interest with a pre-specified amount of a reactant of large molecular size, or magnetized molecule of any size, or both, preferably tethered to a catalyst. The product of the reaction is then filtered out within the blood analysis system and the remaining blood components are returned to the body. This method of “chemical capture” is entirely extracorporeal precluding the use of any ‘drug’ and thereby eliminating the possibility of attendant adverse effects. The chemical capture method of similar nature is employed in specific type of real time PCR where mRNA is separated from reaction mixture using oligodeoxythymidylate [oligo (dT) . . . ]-coated magnetic particles (Ref.11).

In an example implementation, the blood analysis system uses the concept of chemical capture to extract molecules, such as calcium, to prevent coagulation of plasma within the blood analysis system, thereby eliminating the need for anticoagulants, like heparin. The blood analysis system also makes feasible the selective infusion and capture of drugs at different sites of the body. By the use of this technique, a drug may be infused intra-arterially at the site of intended action with prompt removal of the said drug or its metabolites from the venous end in order to limit the adverse effect of the drug and allowing higher doses to be available at the site of action.

### Definitions

All: The word is to be interpreted either in usual sense or it might imply a known fraction with or without other undesirable entities under given set of conditions.

Water: Ultrapure water containing only water molecules or water plus known concentration of molecules and/or ions if latter is necessary for the structural and/or functional stability of the any molecule and/or cells.

Prepared blood/plasma: Suitably diluted blood/plasma so as to reduce the viscosity in such a way that desired flow is achieved, facilitating maximum filtration or separation of detectable.

Solution: Blood/Plasma/plasma minus one type of molecule/plasma minus more than one type of molecule.

Channel: Conduit made up of inert material with controlled gating mechanism and flow rate pumps capable of either facilitating or stopping the flow of solution.

Semi-permeable membrane (SPM): A charged/uncharged membrane with selective pore size allowing passage of specific molecules or group of molecules with specific properties, such as molecular weight (MW), shape and dipole moment (DP), and charge, from an area of high concentration to an area of low or zero concentration. Configuration/design of SPM may vary from unit to unit depending upon the isolation of detectable(s).

Cell: Component consisting of two areas/chambers C1 and C2 divided by an SPM. Both areas/chambers C1 and C2 in a cell C have respective inlets and outlets for solution, water, detectable/s, filtrate and backwash. All the cells are followed by a concentration unit, a detection unit, a correction unit and a holding unit. However, some or all of these can be ignored and the product (both retained and filtered) of one cell can be directed into the input of second unit directly, depending on the logistics.

Unit: A unit may be a cell, a concentration unit, a holding unit, a detection unit, and a correction unit. All inlets and outlets of a unit have separate gates, flow rate pumps, and pressure gauges.

Filtrate: Molecules that have filtered through the SPM from area/chamber C1 to area/chamber C2 and may be suspended in water.

Retentate: Molecules that do not pass through the SPM from area/chamber C1 to area/chamber C2 and may be suspended in water.

Isotonic saline (IS): 0.9% NaCl with physiological pH.

Concentration unit: Unit composed of components to pump out precise amount of water from a given solution. Inlets and outlet of a concentration unit have separate gates, flow rate pumps, and pressure gauges.

Detectable: Entity/entities (molecule(s)/ion(s)) to be detected using a single or combination of detection unit.

Detection unit: Component having source(s) producing and detecting electromagnetic wave(s) and/or field(s) of varying energies and vectors, such that the interaction with detectable(s) does not result in disruption of any kind of interatomic or intramolecular bond. The changes in electromagnetic wave(s) and/or field(s) are directly proportional to the concentration of the detectable. The detection unit is automated and can be programmed to detect and determine quantitative deviation from any set limit or specific concentration. Furthermore, for one or more entities, the detection unit can work in coordination with the correction unit for correcting the concentration of an entity.

Flow rate pumps: Pump meant to ensure a precise rate of flow incorporated at every inlet and outlet throughout a unit and sequential combination of units/cells ensuring the desired flow rate. Flow rate pumps can also be programmed to ensure optimal flow in the entire System such that no part of the system works without coordination with the other parts.

Holding Unit: Area where, if necessary, concentrated detectable is held for a period of time in order to ensure synchronized reconstitution of blood/plasma/serum. Inlets and outlets of a holding unit have separate gates, flow rate pumps, and pressure gauges. Holding units can also be programmed to ensure optimal flow in the blood analysis system such that no part of the blood analysis system works without coordination with the other parts.

Correction Unit: Component having a semi-permeable membrane in which any detectable can be corrected either by extracting the excess detectables using highly selective chemical reaction to capture and remove one or more than one type of molecules and/or ions or by adding the deficient amount of one or more than one type of molecules and/or ions. Correction units can also be programmed to ensure optimal flow in the blood analysis system such that no part of the blood analysis system works without coordination with the other parts. Detection and correction of any entity/entities can be programmed for automated execution.

Capturing molecule: A synthetic molecule (charged/uncharged/magnetized) with tethered and/or magnetized catalyst with extremely high specificity and sensitivity, such that one capturing molecule shall bind with one type of ion/molecule so that the said ion/molecule is ‘captured’ and therefore rendered unfunctional. The capturing molecule typically shall be far larger than the ion/molecule such that only the uncaptured ions/molecules in a given plasma shall be able to pass through the SPM with/without external electromagnetic field and with/without centrifugation, whereas the capturing molecule with or without captured entity shall not be able to pass through. The amount of capturing molecules to be added or the amount of any entity to be removed can be calculated precisely since the preceding detection unit determines the concentration of entity/entities. Therefore, a precise amount of entity/entities of interest can be manipulated.

Collecting channel: A channel into which retentate and/or filtrate of all outlets empty after detection, measurements, and, if necessary, corrections. The collecting channel can also be programmed to ensure optimal flow in the blood analysis system such that no part of the blood analysis system works without coordination with the other parts.

Unit control: Computer-readable instructions controlling the functioning of gates, flow rate pumps, and pressure gauges of all inlets and outlets of a unit, such that there is perfect coordination and continuous flow of solution/detectables.

Cell control: Computer-readable instructions controlling the functioning of gates, flow rate pumps, and pressure gauges of all inlets and outlets of a cell, such that there is perfect coordination and continuous flow of solution/detectables.

Central Control: Computer-readable instructions controlling the functioning of gates, flow rate pumps, and pressure gauges of all inlets and outlets of units, cells, and collecting channels, such that there is perfect coordination and continuous flow all detectable and final reconstitution of blood.

The blood analysis system of the present subject matter is a closed loop extracorporeal system configured to detect and measure in real-time all molecular, ionic and cellular entities in blood without using any chemical reaction that make any alterations to molecular structure unless desired. The system is also configured to extract a precise amount of one or more than one ionic/molecular entity/entities without using any ‘drug’.

In an example, the blood analysis system of the present subject matter is configured to perform the following procedure:
i) draw the blood from a human/animal and direct it into the blood analysis system;
ii) separate all cellular, molecular and/or ionic entities or entities with a particular range of properties using centrifugation and/or sequence of separation and/or filtration units;
iii) direct each of the separated and/or filtered entities to its designated channel;
iv) detect and determine the concentration of each entity using non-chemical reaction methods that neither disrupt any native intramolecular bond nor form any;
v) correcting the concentration of any given ionic or molecular entity/entities using unique concept of chemical capture or using addition of any deficient entity/entities;
vi) detect and determine the concentration of one or more entities post-correction and recorrect if any is not found to be substantially equal to the permissible concentration thereof; and
vii) reconstitute the blood, post-correction (if needed), and infuse the blood back to the human/animal.

In an example, the blood analysis system of the present subject matter is configured to perform the following procedure:
i) draw the blood from a human/animal and direct it into the blood analysis system;
ii) detect and determine the concentration of one or more entities using non-chemical reaction methods that neither disrupt any native intramolecular bond nor form any;
iii) correcting the concentration of any given ionic or molecular entity/entities using unique concept of chemical capture or using addition of any deficient entity/entities;
iv) detect and determine the concentration of one or more entities post-correction and recorrect if any is not found to be substantially equal to the permissible concentration thereof; and
vi) reconstitute the blood, post-correction (if needed), and infuse the blood back to the human/animal.

With the blood analysis system of the present subject matter, particularly using a sequence of an ion separation unit and/or filtration units in the blood analysis system, it is possible to extract any given detectable from blood. The ion separation unit and/or the filtration units composed of semi-permeable membranes (SPMs) may allow only entities with size equal or less than a defined molecular weight cut-off of the SPM.

Once extracted, a single detectable or a mixture of different molecules (solute) in a suitable solvent (water) can be measured by using electromagnetic waves (EMV) or field (EMF) or voltage V of different suitable values/vectors or any other method not preferably but not essentially involving irreversible chemical reaction.

The sequential numbering: first; second; third; fourth; and so on, preceding the terminology used for a component of the blood analysis system may refer to a sequential ordering of the same component appearing in the description herein. The sequential numbering preceding the terminology used for the same components in the description herein may be different from the sequential numbering preceding the terminology of the same components in the claims. Thus, the sequential numbering preceding the terminology does not limit the scope of the claims and is mentioned for the ease of reading and differentiating the same components appearing at different places in the blood analysis system. Also, for one or more components in the description herein, the sequential numbering is not used; however, those components in description may be differentiated by the reference number proceeding the terminology of the same components. Further, for one or more components in the description herein, the sequential numbering is not used in the claims; however, those components may be differentiated by appropriate antecedence-base rule of English grammar.

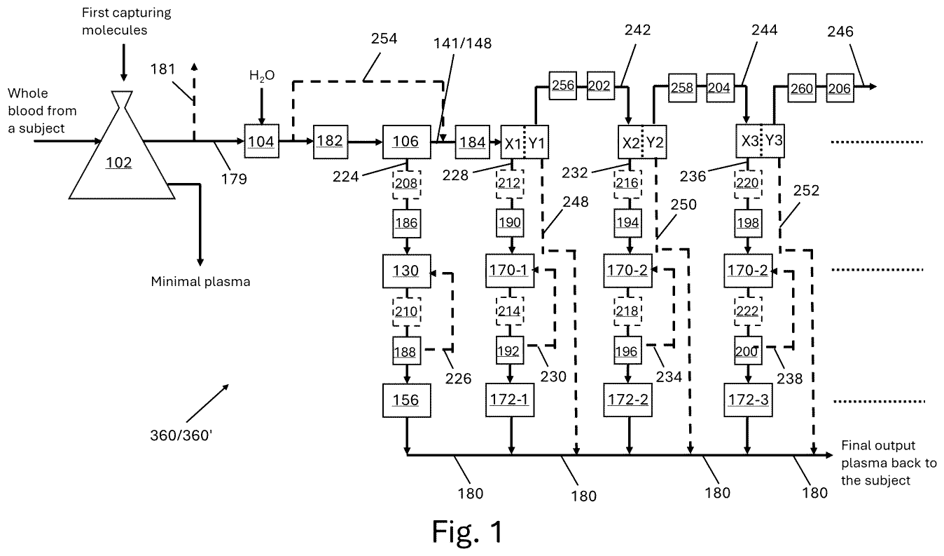

These and other advantages of the present subject matter would be described in a greater detail in conjunction with the FIGS. 1 to 7 in the following description.

It should be noted that the description merely illustrates the principles of the present subject matter. It will thus be appreciated that those skilled in the art will be able to devise various arrangements that, although not explicitly described herein, embody the principles of the present subject matter and are included within its scope. Furthermore, all examples recited herein are intended only to aid the reader in understanding the principles of the present subject matter. Moreover, all statements herein reciting principles, aspects and implementations of the present subject matter, as well as specific examples thereof, are intended to encompass equivalents thereof.

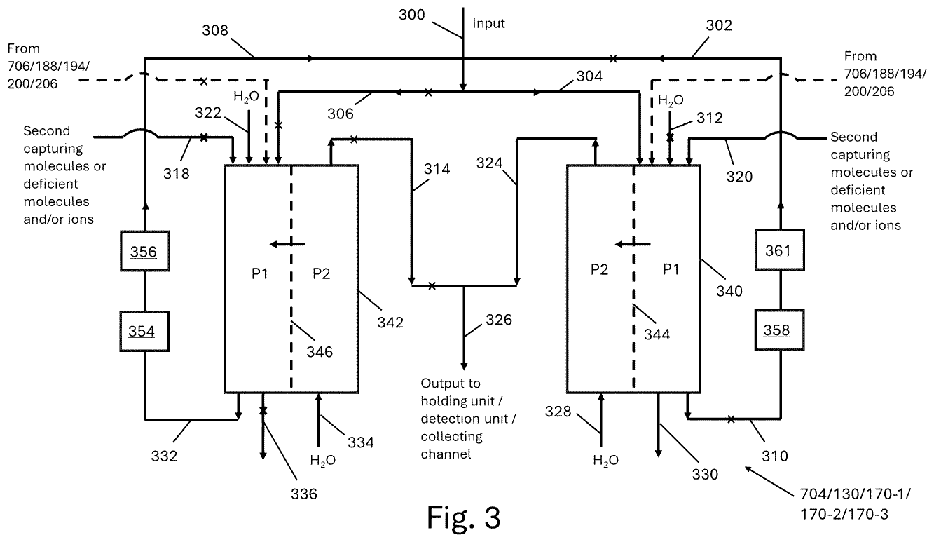

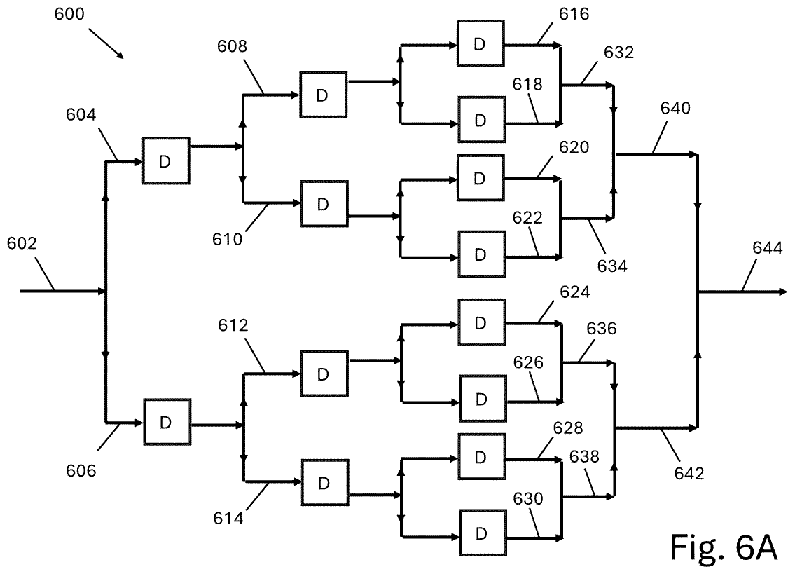

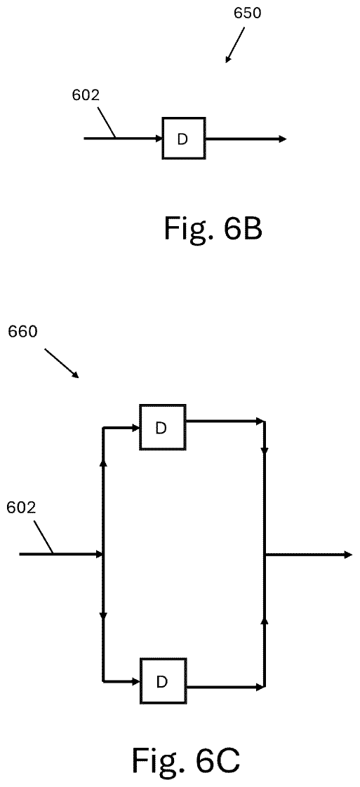

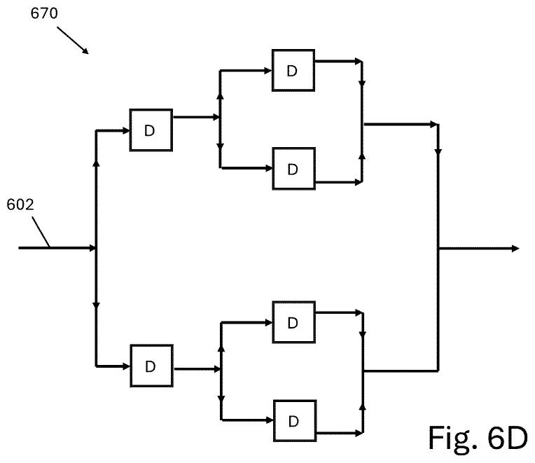

FIG. 1 illustrates an example of a blood analysis system 360, in accordance with the present subject matter. As shown in FIG. 1, whole blood from a subject, e.g., a human or an animal, along with first capturing molecules for capturing any molecules and/or ions, like calcium, that cause coagulation of the blood/plasma, are added and centrifuged in a centrifugation unit 102. As a result of centrifugation, all cellular components are suspended in minimal plasma. The suspended cellular components may subsequently be suspended in an isotonic saline, if necessary, with oxygen and glucose at low temperature. The suspended cellular components, in the minimal plasma, may immediately be infused back to the subject. In an example, platelets, in the minimal plasma, may be separated using an SPM and passed through a channel to a detection unit (explained with reference to FIGS. 6A to 6D) to detect the concentration of the platelets. Further, granulocytes and RBCs, in the minimal plasma, may be separated using ultracentrifugation, directed to two different channels, and their concentrations are detected by suitable detection units (not shown in FIG. 1). Further, WBCs+RBCs+platelets may be passed through a channel, such that there are no shadow areas and detection units (not shown in FIG. 1) may detect the type of cellular components and determine their concentrations. The detection unit includes a spectroscopy-based measurement unit, an optical sensing unit, a non-chemical sensing unit, an electrochemical sensing unit, a chemical reaction-based measurement unit, or a combination thereof. In an example, concentration of a specific cellular component may be determined by signature scattering of suitable wavelengths. Any or all from the platelets, the granulocytes, the RBCs, etc., in the minimal plasma, that may be separated and detected may be corrected using a correction unit (explained with reference to FIG. 3) before infusing the minimal plasma with the corrected concentrations of the platelets, the granulocytes, the RBCs, etc., to the subject.

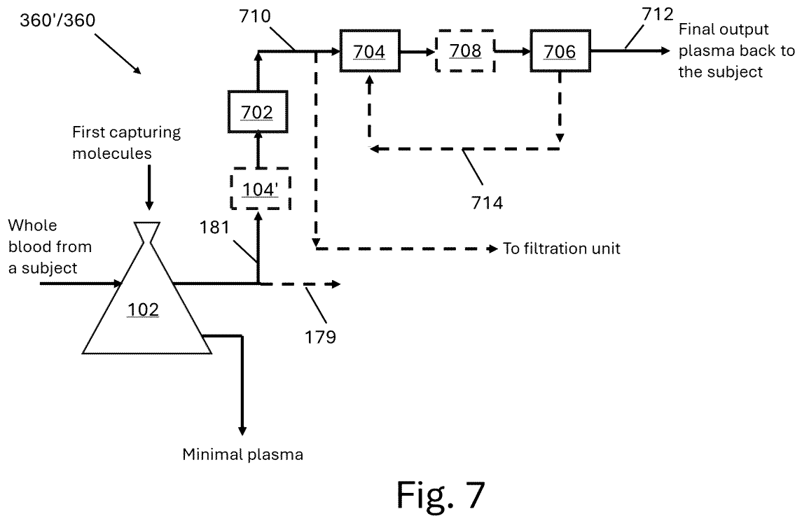

The blood analysis system 360 includes a controlled gating mechanism to flow the plasma from the centrifugation unit 102 either to a channel 179 into a holding unit 104 or to a channel 181 (described in detail with reference to FIG. 7). In the holding unit 104, the plasma may be diluted with water, if necessary, to adjust viscosity of the plasma such that suitable flow of the plasma is achieved facilitating maximum detection, separation, and/or filtration, as the case may be, at subsequent stages of the blood analysis system 360.

From the holding unit 104, the plasma with remaining number of the first capturing molecules and unbound molecules and ions, but without the cellular components is passed to a detection unit 182 to detect, and determine concentration of, cations and/or anions in the plasma. After determining the concentration of the cations and/or anions, the plasma is passed to an ion separation unit 106, where anions and cations from the plasma are separated. The anions and the cations may be separated or isolated using the ion separation unit 106 in either order, i.e., the anions before the cations, or the cations before the anions.

### Configuration of the Ion Separation Unit 106;

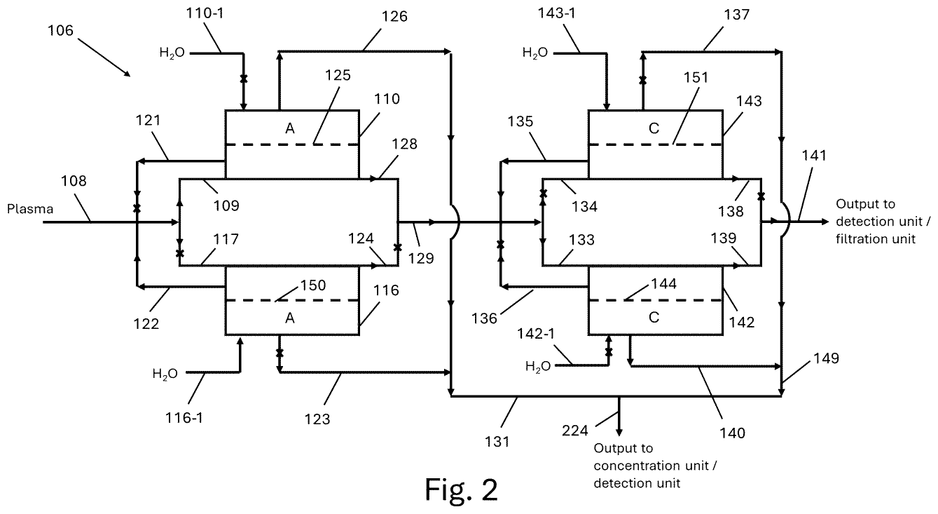

FIG. 2 illustrates an example of an ion separation unit 106, in accordance with the present subject matter. As shown in FIG. 2, the plasma from the holding unit 104 is passed through channel 108 towards channel 109 and then to a cell 110. For said flow of plasma to happen, channels 117, 121, 123 and 124 and 110-1 are kept closed via a respective valve, (every entry/exit of channel/cell has a valve) and water is passed to cell 116 via channel 116-1 to declog SPM 150. The declogged matter is passed to channel 122 which in turn empties into channel 108. The cell 110 has an SPM 125 which allows only anions to pass through. The filtered anions (the filtrate) from the cell 110 are passed through channels 126 and 131 to an output channel 224, and the remaining plasma (anion-free plasma) from the cell 110 is passed via channels 128 and 129 to a further unit for separation of cations, as explained below.

The plasma (anion-free plasma) from channel 129 is passed towards channel 133 and then to a cell 142. For said flow of plasma to happen, channels 134, 136, 137 and 138 and 142-1 are kept closed, and water is passed to cell 143 via channel 143-1 to declog SPM 151. The declogged matter is passed to channel 135 which in turn empties into channel 129. The cell 142 has an SPM 144 which allows only cations to pass through. The cations (the filtrate) from the cell 142 are passed through channels 140 and 149 to an output channel 224, and the remaining plasma (anion- and cation-free plasma) from the cell 142 is passed via channel 139 to channel 141.

After a while, depending on the properties of SPM and the molecular load, the SPM may get clogged. Therefore, it is essential to declog the SPM. Since one of the mandates of the blood analysis system 360 is to have a continuous and real-time measurements and corrections of different entities, declogging mechanism while maintaining continuous flow is of paramount importance.

The solution offered is in the form of two parallel identical units such that while one of them if filtering and producing detectable, the other one is getting declogged. This aspect is depicted in FIG. 2, where while the unit 110 is filtering the anions the unit 116 is getting declogged. Each of the units 110 and 116 has a respective SPM 125 and 150, which can allow only anions to pass through. For using the cell 110 for filtering anions and declogging the cell 116, the channels 117, 121, 123, and 124 and 110-1 are kept closed, the channels 109, 122, 126, 128 are kept open, and water is passed through the cell 116 via channel 116-1 to declog SPM 150. The declogged matter is passed to channel 122 which in turn empties into channel 108. Alternately, for using the cell 116 for filtering anions and declogging the cell 110, the channels 117, 121, 123, and 124 and 110-1 are kept open, the channels 109, 122, 126, 128 and 116-1 are kept closed, and water is passed through the cell 110 via channel 110-1. The declogged matter is passed to channel 121 which in turn empties into channel 108. The anions, filtered by the SPM 150 (the filtrate), from the cell 116 are passed through channels 123 and 131 to the output channel 224, and the remaining plasma (anion-free plasma) from the cell 116 is passed via channels 124 and 129 to a further unit for separation of cations, as explained earlier.

Similarly, as depicted in FIG. 2, while the unit 142 is filtering the cations the unit 143 is getting declogged. Each of the units 142 and 143 has a respective SPM 144 and 151, which can allow only cations to pass through. For using the cell 142 for filtering cations and the cell 143 for declogging, the channels 134, 136, 137, and 138 and 142-1 are kept closed, the channels 133, 135, 139, 140 and 143-1 are kept open, and water is passed through the cell 143 via channel 143-1. The declogged matter is passed to channel 135 which in turn empties into channel 129. Alternately, for using the cell 143 for filtering cations and declogging the cell 142, the channels 134, 136, 137, and 138 and 142-1 are kept open, the channels 133, 135, 139, 140 and 143-1 are kept closed, and water is passed through the cell 142 via channel 142-1. The declogged matter is passed to channel 136 which empties into channel 129. The cations, filtered by the SPM 151 (the filtrate), from the cell 143 are passed through channels 137 and 149 to the output channel 224, and the remaining plasma (anion- and cation-free plasma) from the cell 143 is passed via channels 138 to the channel 141.

FIG. 2 illustrates the ion separation unit 106 in which the anions are separated before the cations. In an example where the cations are to be separated before the anions, the cells 110 and 116 have a respective SPM which allows only cations to pass through and the cells 142 and 143 have a respective SPM which allows only anions to pass through.

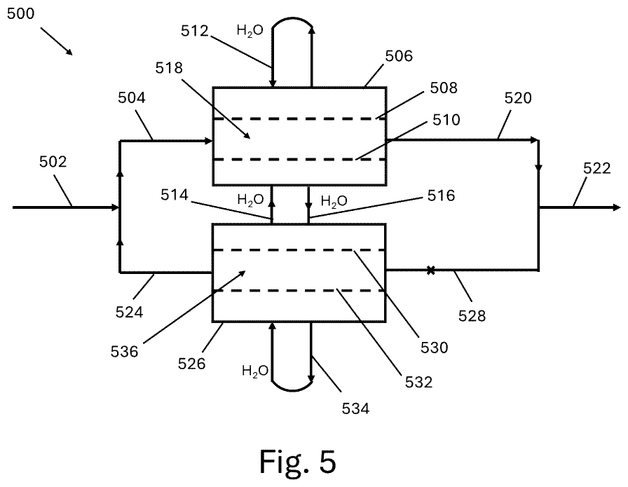

Returning to FIG. 1, the cations and anions from the output channel 224 are passed to a concentration unit 208 and a detection unit 186 for concentrating the cations and anions and detecting, and determining concentrations of, the cations and anions, respectively. The configuration and operation of the concentration unit and the detection unit are described later with reference to FIG. 5 and FIG. 6, respectively. The cations and anions from the detection unit 186 are passed to a correction unit 130, as shown in FIG. 1. The configuration and operation of the correction unit are described later with reference to FIG. 3. The correction unit 130 is operated to either pump in extra cations and/or anions if the concentration of any is determined to be less than permissible concentration or extract out the extra amount of cations and/or anions if the concentration of any is determined to be more than the permissible concentration. Further, in the blood analysis system 360, a concentration unit 210 (optional) and a detection unit 188 are coupled to the correction unit 130. The detection unit 188 is operated to detect, and determine concentration of, the corrected cations and/or anions from the correction unit 130. In response to determining the concentration of the cations and/or anions output from the correction unit 130 not equal to permissible concentration, the blood analysis system 360 is operated to operate the detection unit 188 to flow the cations and anions back to the correction unit 130 through channel 226, where the correction unit 130 is further operated to recorrect the concentration of the cations and/or anions. After necessary correction, the cations and/or anions are held in a holding unit 156 which in turn empties into a collecting channel 180.

Although FIG. 1 shows a single output channel 224 for carrying both cations and anions and passing them to a single concentration unit 208, a single detection unit 186, a single correction unit 130 with a single channel for re-correction, a single holding unit 156 for both cations and anions; however, in an example, the ion separation unit 106 may have separate output channels for cations and anions and therefore separate concentration units, detection units, correction units with a respective channel for re-correction, and holding units, for each of cations and anions with both emptying separately into the collecting channel 180.

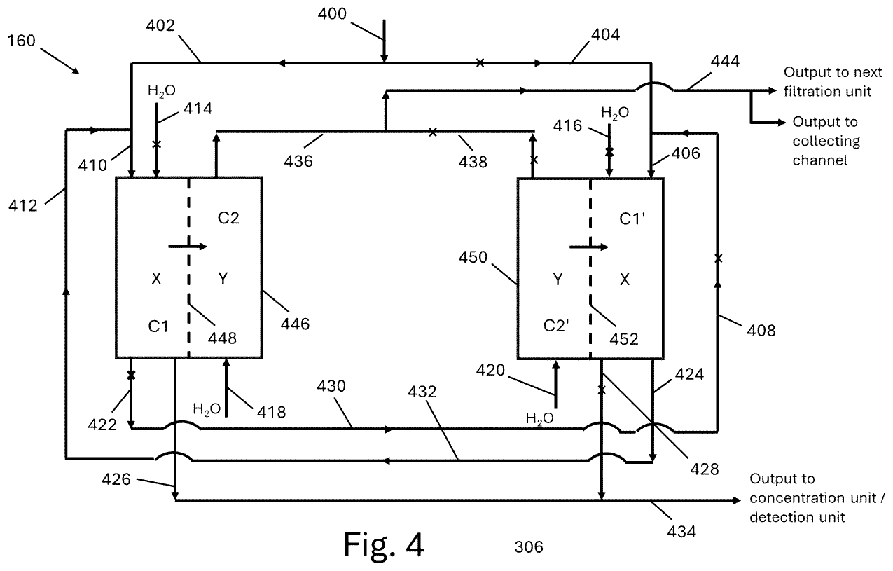

Further, as shown in FIG. 1, suitably diluted ion-free plasma from channel 141 of the ion separation unit 106 is then passed through a first filtration unit 160-1 to filter out a first specific type of molecules X1 from the ion-free plasma. The configuration and operation of a filtration unit is described later in the description with reference to FIG. 4. The first specific type of molecules X1, filtered out from the first filtration unit 160-1, suspended in water after concentration, by a concentration unit 212, and detection, by a detection unit 190, are directed through channel 228 to a correction unit 170-1 in order to either pump in extra molecules of the first specific type if the levels are low or extract out the exact amount of molecules of the first specific type if the levels are high. The configuration and operation of the concentration unit, the detection unit, and the correction unit are described later with reference to FIG. 5, FIG. 6, and FIG. 3, respectively. The correction unit 170-1 is operated to either pump in extra molecules of the first specific type if their concentration is determined to be less than permissible concentration or extract out the extra molecules of the first specific type if their concentration is determined to be more than the permissible concentration. Further, in the blood analysis system 360, a concentration unit 214 (optional) and a detection unit 192 are coupled to the correction unit 170-1. The detection unit 192 is operated to detect, and determine concentration of, the corrected first specific type of molecules from the correction unit 170-1. In response to determining the concentration of the first specific type of molecules output from the correction unit 170-1 not equal to permissible concentration, the blood analysis system 360 is operated to operate the detection unit 192 to flow the first specific type of molecules back to the correction unit 170-1 through channel 230, where the correction unit 170-1 is further operated to recorrect the concentration of the first specific type of molecules. After necessary correction, the first specific type of molecules X1 are held in a holding unit 172-1 which in turn empties into the collecting channel 180.

The plasma with molecules Y1 (filtrate Y1 shown in FIG. 1) from the first filtration unit 160-1, suspended in water after concentration, by a concentration unit 256, and detection, by a detection unit 202, are directed through channel 242 to a second filtration unit 160-2 to filter out a second specific type of molecules X2. The second specific type of molecules X2, filtered out from the second filtration unit 160-2, suspended in water after concentration, by a concentration unit 216, and detection, by the detection unit 194, are directed through channel 232 to a correction unit 170-2 in order to either pump in extra molecules of the second specific type if the levels are low or extract out the exact amount of molecules of the second specific type if the levels are high. The configuration and operation of the concentration unit, the detection unit, and the correction unit are described later with reference to FIG. 5, FIG. 6, and FIG. 3, respectively. The correction unit 170-2 is operated to either pump in extra molecules of the second specific type if their concentration is determined to be less than permissible concentration or extract out the extra molecules of the second specific type if their concentration is determined to be more than the permissible concentration. Further, in the blood analysis system 360, a concentration unit 218 (optional) and a detection unit 196 are coupled to the correction unit 170-2. The detection unit 196 is operated to detect, and determine concentration of, the corrected second specific type of molecules from the correction unit 170-2. In response to determining the concentration of the second specific type of molecules output from the correction unit 170-2 not equal to permissible concentration, the blood analysis system 360 is operated to operate the detection unit 196 to flow the second specific type of molecules back to the correction unit 170-2 through channel 234, where the correction unit 170-2 is further operated to recorrect the concentration of the second specific type of molecules. After necessary correction, the second specific type of molecules X2 are held in a holding unit 172-2 which in turn empties into the collecting channel 180. In an example, the plasma with molecules Y1 (filtrate Y1 shown in FIG. 1) from the first filtration unit 160-1 is passed through channel 248 to the collecting 180 without any further filtration.

The plasma with molecules Y2 (filtrate Y2 shown in FIG. 1) from the second filtration unit 160-2, suspended in water after concentration, by a concentration unit 258, and detection, by a detection unit 204, are directed through channel 244 to a third filtration unit 160-3 to filter out a third specific type of molecules X3. The third specific type of molecules X3, filtered out from the third filtration unit 160-3, suspended in water after concentration, by a concentration unit 220, and detection, by the detection unit 198, are directed through channel 236 to a correction unit 170-3 in order to either pump in extra molecules of the third specific type if the levels are low or extract out the exact amount of molecules of the third specific type if the levels are high. The configuration and operation of the concentration unit, the detection unit, and the correction unit are described later with reference to FIG. 5, FIG. 6, and FIG. 3, respectively. The correction unit 170-3 is operated to either pump in extra molecules of the third specific type if their concentration is determined to be less than permissible concentration or extract out the extra molecules of the third specific type if their concentration is determined to be more than the permissible concentration. Further, in the blood analysis system 360, a concentration unit 222 (optional) and a detection unit 200 are coupled to the correction unit 170-3. The detection unit 200 is operated to detect, and determine concentration of, the corrected third specific type of molecules from the correction unit 170-3. In response to determining the concentration of the third specific type of molecules output from the correction unit 170-3 not equal to permissible concentration, the blood analysis system 360 is operated to operate the detection unit 200 to flow the third specific type of molecules back to the correction unit 170-3 through channel 238, where the correction unit 170-3 is further operated to recorrect the concentration of the third specific type of molecules. After necessary correction, the third specific type of molecules X3 are held in a holding unit 172-3 which in turn empties into the collecting channel 180. In an example, the plasma with molecules Y2 (filtrate Y2 shown in FIG. 1) from the first filtration unit 160-2 is passed through channel 250 to the collecting 180 without any further filtration.

The plasma with molecules Y3 (filtrate Y3 shown in FIG. 1) from the third filtration unit 160-3, suspended in water after concentration, by a concentration unit 260, and detection, by a detection unit 206, through channel 246 may either be directed to another filtration unit (not shown) to filter out a further specific type of molecules from the plasma with molecules Y3, or directed to the collection channel 180 via channel 252.

In an example implementation, the blood analysis system 360 may include N number of filtration units to filter out N number of specific types of molecules from the ion-free plasma, N is equal to zero or more than zero. One filtration unit is ideally configured to filter out one specific type of molecules, but more than one type of molecules with given electrochemical and physical properties can also be filtered out provided accurate detection system to measure the concentrations of the same is in place. Each of the filtration units of the blood analysis system 360 may be configured to operate in the same manner. The configuration and operation of a filtration unit is described later in the description with reference to FIG. 4.

### Configuration of the Correction Unit 130/132/170-1/170-2/170-3:

The proposed principle for selective removal of cellular or molecular or ionic entities is very specific chemical reaction, in which the reaction product in aqueous medium will be retained in the unit (i.e., reaction products do not pass through the SPM of the unit) owing to the cellular/molecular/ionic weight, and/or size and/or charge, whereas rest of the cellular or molecular and ionic entities can pass through the SPM. The amount of the entity to be removed can be controlled as the concentration of the entities is already known by the prior detection unit.

Removal of any molecular/ionic entity requires engineering of a capturing molecule which would bind or capture only one type of entity (molecule or ion). Since such chemical reaction may require a catalyst, the catalyst itself can be incorporated or tethered onto the capturing molecule thereby making separate removal of catalyst from the circulation unnecessary. Alternatively, the catalyst itself can be separate but magnetized or charged to facilitate the subsequent removal via application of suitable magnetic field on the SPM.

At present elevated levels of Sodium, or Potassium, or Calcium, etc., require therapeutic maneuvers demanding extreme care including management of adverse effects. Chemical Capture of such entities using capturing molecules can remove excess amount of any entity without any drug. It can be any number of types of cellular/ionic/molecular entities that are logistically possible to handle. The definition of capturing molecules is included under the Definitions mentioned above.

FIG. 3 illustrates an example of a correction unit, in accordance with the present subject matter. Plasma with cations and/or anions from the ion separation unit 106 or plasma with the first capturing molecules and one or more specific types of molecules and/or ions from a filtration unit, as the case may be, is received in the correction unit via channel 300.

A: For Removal of Excess Entities (Cations or Anions or Specific Type of Molecules and/or Ions)

Plasma enters the correction unit via channel 300. As shown in FIG. 3, the plasma from the channel 300 moves towards channel 304 and then to a cell 340. For said flow of plasma to happen, channels 302, 306, 310, 312, 314, 316, 318, and 336 are kept closed, channels 304, 308, 320, 322, 324, 326, 328, 330, 332, and 334 are kept open, and water is passed to cell 342 via channels 322 (to chamber on a retentate side P1 of 342) and via 334 (to chamber on a filtrate side P2 of 342). The declogged matter from the chamber on the retentate side P1 of the cell 342 passes out via channel 332 which in turn after concentration, by a concentration unit 354, and detection, by a detection unit 356, continues in channel 308 and finally empties into 300. The cell 340 has an SPM 344. Second capturing molecules enter a chamber on a retentate side P1 of the cell 340 through channel 320. The concentration of the second capturing molecules depend on the amount of excess entities detected by a detection unit preceding the correction unit and by the detection unit 356 and to be removed from the plasma. The first capturing molecules and the second capturing molecules with entities captured thereto do not pass through the SPM 344 and are thus retained in the chamber of the retentate side P1 of the cell 340 and exited through channel 330. Channel 330 carrying the first and second capturing molecules bound with entities empty into a waste chamber (not shown in FIG. 3). Minimal but suitable water is passed through a chamber on a filtrate side P2 of the cell 340 from channel 328. The uncaptured entities get filtered to the chamber on the filtrate side P2 of the cell 340 and exited from the chamber P2 of the cell 340 via channel 324 towards an output channel 326. The output from the output channel 326 is passed to a concentration unit (optional) and to a detection unit for rechecking and re-correction, if required, before being directed to a holding unit which in turn empties into the collecting channel 180, as described earlier in the description. The amount of entities bound with the first capturing molecules, which are discarded, are, for example, either added back to the plasma in the collecting channel 180 before infusing the plasma back to the subject or administered to the subject independently and not from the blood analysis system.

### B: For Correction of Low Concentration of any Entity

Plasma enters the correction unit via channel 300. As shown in FIG. 3, the plasma from the channel 300 moves towards channel 304 and then to a cell 340. For said flow of plasma to happen, channels 302, 306, 310, 312, 314, 316, 318, and 336 are kept closed, channels 304, 308, 320, 322, 324, 326, 328, 330, 332, and 334 are kept open, and water is passed to cell 342 via channel 322 (to chamber on a retentate side P1 of 342) and via channel 334 (to chamber on a filtrate side P2 of 342). The declogged matter from the chamber on the retentate side P1 of the cell 342 passes out via channel 332 which in turn after concentration, by a concentration unit 354, and detection, by a detection unit 356, continues in channel 308 and finally empties into 300. The cell 340 has an SPM 344. The deficient amount of molecule(s) and/or ion(s) enters a chamber on a retentate side P1 of the cell 340 through channel 320. The concentration of the deficient amount of molecule(s) and/or ion(s) depends on the concentration of molecule(s) and/or ion(s) detected by a detection unit preceding the correction unit and by the detection unit 356. Minimal but suitable water is passed through a chamber on a filtrate side P2 of the cell 340 from channel 328. The uncaptured entities get filtered to the chamber on the filtrate side P2 and exited from the chamber P2 via channel 324 towards an output channel 326. The output from the output channel 326 is passed to a concentration unit (optional) and to a detection unit for rechecking and re-correction, if required, before being directed to a holding unit which in turn empties into the collecting channel 180, as described earlier in the description. The first capturing molecules with entities captured thereto do not pass through the SPM 344 and are thus retained in the chamber of the retentate side P1 of the cell 340 and exited through channel 330. Channel 330 carrying first capturing molecules bound with entities empty into waste chamber (not shown in FIG. 3). The amount of entities bound with the first capturing molecules, which are discarded, are, for example, either added back to the plasma in the collecting channel 180 before infusing the plasma back to the subject or administered to the subject independently and not from the blood analysis system.

As described for the ion separation unit 106, the SPM 344 of the cell 340 may get clogged, depending on the properties of the SPM 344 and the molecular load. As shown in FIG. 3, the correction unit also has two parallel identical cells 340 and 342, such that while one of them is filtering, the other one is getting declogged. Each of the units 340 and 342 has a respective SPM 344 and 346. For using the cell 340 for filtering and for declogging the cell 342, the channels 302, 306, 310, 312, 314, 316, 318, and 336 are kept closed, channels 304, 308, 320, 322, 324, 326, 328, 330, 332, and 334 are kept open, and water is passed to cell 342 via channel 322 (to chamber on a retentate side P1 of 342) and via 334 (to chamber on a filtrate side P2 of 342) such that the pressure in the chamber on a filtrate side P2 of the cell 342 is greater than chamber on a retentate side P1 of the cell 342. The declogged matter from the retentate side P1 of the cell 342 exits via channel 332 which continues to channel 308 which in turn empties into channel 300. Alternately, for using the cell 342 for filtering and for declogging the cell 340, the channels 302, 306, 310, 312, 314, 316, 318, and 336 are kept open, channels 304, 308, 320, 322, 324, 326, 328, 330, 332, and 334 are kept closed, and water is passed to cell 340 via channel 312 (to chamber on a retentate side P1 of 340) and via 328 (to chamber on a filtrate side P2 of 340) such that pressure in the chamber on a filtrate side P2 of the cell 340 is greater than the chamber on a retentate side P1 of 340. The declogged matter from the chamber on a retentate side P1 of 340 exits via channel 310 which after concentration, by a concentration unit 358, and detection, by a detection unit 361, continues to 302 and which in turn empties into channel 300. Like for the cell 340, the uncaptured entities get filtered to the chamber on the filtrate side P2 of the cell 342 and exited from the chamber P2 of the cell 342 via channel 314 towards an output channel 326. The output from the output channel 326 is passed to a concentration unit (optional) and to a detection unit for rechecking and re-correction, if required, before being directed to a holding unit which in turn empties into the collecting channel 180, as described earlier in the description.

### Configuration of the Filtration Unit 160-1/160-2/160-3/ . . . (Individually Referred to as the Filtration Unit 160):

FIG. 4 illustrates an example of a filtration unit 160, in accordance with the present subject matter. The plasma with remaining number of the first capturing molecules and unbound molecules and ions, but without the cellular components from the holding unit 104 via channel 254 and then from the detection unit 184, or the plasma with remaining number of the first capturing molecules and unbound molecules and ions, but without the cellular components from the channel 710 (as described with respect to FIG. 7), or the ion free plasma from the ion separation unit 106 and the detection unit 184, or the plasma, i.e., the filtrate Y1, Y2, Y3, or so on, from a previous filtration unit, as the case may be, is received via channel 400 of the filtration unit 160, as shown in FIG. 4.

As shown in FIG. 4, the plasma from the channel 400 moves towards channel 402 and then to a cell 446. For said flow of plasma to happen, channels 404, 406, 408, 414, 422, 428, 430, 438 and 442 are kept closed, channels 400, 402, 410, 412, 418, 424, 426, 432, 434, 436, 440, and 444 are kept open. The plasma is passed to a chamber C1 on a retentate side of the cell 446 via the channel 410 and water is passed through a chamber C2 on the filtrate side of the cell 446 from channel 418. The detectables X exit the chamber C1 of the cell 446 via the channel 426 to an output channel 434 whereas the filtrate Y exits the chamber C2 of the cell 446 via the channel 436 to an output channel 444. Water is passed via the channel 416 to a chamber C1′ on a retentate side of the cell 450 and via the channel 420 to a chamber C2′ on a filtrate side of the cell 450 such that the pressure in the chamber C2′ of the cell 450 is higher than the chamber C1′ of the cell 450 so that clogging matter on a SPM 452 of the cell 450 is declogged and exits the chamber C1′ of the cell 450 via the channel 424. The channel 424 continues to the channel 432 which is continuous to the channel 412 which finally empties into the chamber C1 of the cell 446 via the channel 410. The cell 446 has an SPM 448. The SPM 448 is such that molecules X (e.g., X1, X2, X3, and so on, as the case may be) do not pass through the SPM 448 and the remaining molecules Y (Y1, Y2, Y3, and so on, as the case may be) in the plasma pass through the SPM 448. The molecules X are thus retained in the plasma entering the chamber C1 of the cell 446 and exit through the channel 426 to the output channel 434. The contents of the channel 434 are concentrated, detected and corrected, as described earlier, before emptying in the collecting channel 180. Minimal but suitable water is passed through the chamber C2 of the cell 446 via the channel 418 such that pressure and concentration gradient in the chamber C1 of the 446 is higher than the chamber C2 of the cell 446 so that the filtrate Y across the SPM 448 exits the chamber C2 of the cell 446 via the channel 436 to the output channel 444. The output from the channel 444 is passed to a next filtration unit or to the collecting channel 180, as the case may be.

As described for the ion separation unit 106 and for the correction unit, the SPM 448 of the cell 446 may get clogged, depending on the properties of the SPM 448 and the molecular load. As shown in FIG. 4, the filtration unit also has two parallel identical cells 446 and 450, such that while one of them if filtering, the other one is getting declogged. Each of the cells 446 and 450 has a respective SPM 448 and 452. For using the cell 446 for filtering and the cell 450 for declogging, the channels 404, 406, 408, 414, 422, 428, 430, 438 and 442 are kept closed, the channels 400, 402, 410, 412, 418, 424, 426, 432, 434, 436, 440, and 444 are kept open and water is passed to the cell 450 via the channel 416 to the chamber C1′ of the cell 450 and via the channel 420 to the chamber C2′ of the cell 450 such that the pressure in the chamber C2′ is higher than that in the chamber C1′ so that clogging matter on the SPM 452 is declogged and exits the chamber C1′ via the channel 424. Alternately, for using the cell 450 for filtering and for declogging the cell 446, the channels 404, 406, 408, 414, 422, 428, 430, 438 and 442 are opened and the channels 400, 402, 410, 412, 418, 424, 426, 432, 434, 436, 440, and 444 are kept closed. Water is passed to the chamber C2′ of the cell 450 via the channel 420 and the plasma enters the chamber C1′ of the cell 450 via the channel 406 such that because of pressure and concentration gradient along with properties of SPM 452, the resulting filtrate leaves the chamber C2′ via the channel 438 to the output channel 444. The detectables retained in the chamber C1′ exits via the channel 428 to the output channel 434. Meanwhile, water enters the chamber C2 of the cell 446 via the channel 418 and the chamber C1 of the cell 446 via the channel 414 such that the pressure in the chamber C2 is higher than that in the chamber C1 so that the clogging matter on SPM 448 in the chamber C1 side is dislodged and the same exits the chamber C1 via the channel 422 which continues to channel 430 which continues to channel 408 and which finally empties into the channel 406. The output from the channel 444 to a next filtration unit or to the collecting channel 180, as the case may be. The contents of the channel 434 are concentrated, detected and corrected, as described earlier, before emptying in the collecting channel 180.

### Configuration of the Concentration Unit:

FIG. 5 illustrates an example of a concentration unit 500, in accordance with the present subject matter. In the concentration unit 500, a precise amount of water is pumped out of the input plasma, i.e., the plasma that enters the concentration unit 500.

The plasma with molecules suspended in water enters the concentration unit 500 via channel 502. The input plasma moves towards a cell 506 via channel 504. In the cell 506, there is a symmetrical arrangement of SPMs 508 and 510, as shown in FIG. 5 such that only water from the cell is able to pass through the SPMs 508 and 510 to the exit channels 514 and 516.

The high but variable/controllable osmotic pressure is generated by high concentration of suitable molecules such that the said molecules are confined only and only to the area between SPMs 508 and 510 outer walls of unit 506. Similarly, the said molecules are confined only and only to the area between SPMs 530 and 532 outer walls of unit 526. In addition, a suitable negative pressure can be applied at exit channels 512/514/516 to expedite removal of water. The removed water can be circulated within the concentration unit 500 or to another unit of the blood analysis system 360. The concentrated plasma from the area 518 of the cell 506 exits via channels 520 and 522, and moves to a further unit, as the case may be. Similar to other units with SPMs an identical unit is placed parallel to 506 which is 526 as shown in the figure such that when 506 is functioning as primary unit for the removal of water unit 526 is getting declogged which is facilitated by closure of channel 528 and pumping of water into 526 such that clogging material on SPMs 530 and 532 is dislodged and collected in 536 from where it is routed back to the primary unit 506 via channel 524.

### Configuration of the Detection Unit:

With the ultimate aim of separating the cellular and/or the molecular and/or the ionic entities in blood and measuring the concentration of one or more of those in real time in a manner keeping the said entities suitable for reinfusion back to the subject after combining them back again to reconstitute plasma/serum or whole blood, other approaches can be utilized in addition to SPM either in isolation or various combinations. The other approaches may include the following:
i) Centrifugation including ulracentrifugation (Centri).
ii) EMF of varying strengths at different stages (EMF).
   Therefore, the combinations may be (a) SPM+Centri+EMF, (b) SPM+EMF; and (c) EMF+Centri.

A detection unit is used for measuring the concentration of any one type of molecules. The molecules are suspended in water. Let water flow in a channel with a diameter ‘d’ at a flow rate ‘r’. The detecting unit comprises: a source of electromagnetic waves (EMV) or field (EMF) or voltage V of different suitable values; and a detector located opposite to the source measuring changes in either EMV, EMF or V. The changes are going to be a function of the number of water molecules, the flow rate ‘r’, and the diameter ‘d’ of the channel. The changes under given conditions can be quantified.

Let's assume now that there are N number of single type molecules uniformly distributed in a given volume of water flowing past the detection unit outlined above. The changes in EMV, EMF or V now are going to be different which in turn are going to be function of concentration of single type of molecules. The changes and corresponding concentrations can be standardized.

Any non-chemical method such as ultraviolet/infrared/visible spectroscopy, absorbance, transmission, impedence, conductivity, selective lasers or combinations of any of these as well as other similar methods can be utilized as long as no structural change is made to the detectable/s. Or even, if any changes are made those ideally be reversible. In an example, any chemical and electrochemical techniques can be utilized.

The last option would be chemical reaction; however, it too must be completely reversible. Since the detectable/s, to be detected, are suspended in water and in continuous flow, the said detectable/s are likely to be highly diluted and therefore it may be challenging to measure the concentration by using methods mentioned above.

The solution would be to ‘concentrate’ the detectable using the concentration unit, whereby a suitable amount of water is removed. One can then use an appropriate spectroscopic technique such as magnetic resonance or laser spectroscopy or IR or UV or a combination of them, to obtain the relative concentrations or percentages of molecules depending upon the knowledge about the types of molecules present with in the solution. Relative concentrations of different entities are not going to yield the absolute values. Therefore, before detection, a known quantity of: (a) inert molecule may be added; or (b) a molecule with no adverse effect to the patient may be added; or (c) a molecule with an adverse effect to the patient may be added and subsequently chemically captured and removed. The relative percentages of different molecules can be converted into absolute values based on the known concentration of introduced entity.

FIG. 6A illustrates an example of a detection unit 600, in accordance with the present subject matter. The underlying principle to reduce the requirement of complex algorithms would be to reduce the number and/or types of molecules. This can be achieved by progressive bifurcation of the main channel 602 carrying the detectable with bifurcated channels 604, 606, . . . , each of the two bifurcated channels having equal probability of receiving molecular load from the input channel. Thus, each of the two bifurcated channels carry half or 50% load of the original amount being carried by the input channel. A spectroscopy unit is placed at each of the channels 602, 604, 606, such that concentration of molecules in each channel can be calculated in relation to the molecules in any other channel. Post detection the channels can converge as shown in FIG. 6A. The number of bifurcations may be directly proportional to the molecular load and the number of types of molecules. The number of bifurcations may also likely to be dictated by sensitivity of detectors. The detection unit 600 of FIG. 6A illustrates three levels of bifurcation and convergence channels.

FIG. 6B illustrates an example of a detection unit 650, in accordance with the present subject matter. The detection unit 602 of FIG. 6B illustrates a single channel-based detection unit, without any bifurcation and convergence channels.

FIG. 6C illustrates an example of a detection unit 660, in accordance with the present subject matter. The detection unit 604 of FIG. 6C illustrates one level of bifurcation and convergence channels.

FIG. 6D illustrates an example of a detection unit 670, in accordance with the present subject matter. The detection unit 606 of FIG. 6D illustrates two levels of bifurcation and convergence channels.

FIG. 7 illustrates an example of a blood analysis system 360′, in accordance with the present subject matter. As shown in FIG. 7, whole blood from a subject, e.g., a human or an animal, along with first capturing molecules for capturing any molecules and/or ions, like calcium, that cause coagulation of the blood/plasma, are added and centrifuged in a centrifugation unit 102, similar to that of the blood analysis system 360. As a result of centrifugation, all cellular components are suspended in minimal plasma. The suspended cellular components may subsequently be suspended in an isotonic saline, if necessary, with oxygen and glucose at low temperature. The suspended cellular components, in the minimal plasma, may immediately be infused back to the subject. In an example, platelets, in the minimal plasma, may be separated using an SPM and passed through a channel to a detection unit (explained with reference to FIGS. 6A to 6D) to detect the concentration of the platelets. Further, granulocytes and RBCs, in the minimal plasma, may be separated using ultracentrifugation, directed to two different channels, and their concentrations are detected by suitable detection units (not shown in FIG. 7). Further, WBCs+RBCs+platelets may be passed through a channel, such that there are no shadow areas and detection units (not shown in FIG. 7) may detect the type of cellular components and determine their concentrations. The detection unit includes a spectroscopy-based measurement unit, an optical sensing unit, a non-chemical sensing unit, an electrochemical sensing unit, a chemical reaction-based measurement unit, or a combination thereof. In an example, concentration of a specific cellular component may be determined by signature scattering of suitable wavelengths. Any or all from the platelets, the granulocytes, the RBCs, etc., in the minimal plasma, that may be separated and detected may be corrected using a correction unit (explained with reference to FIG. 3) before infusing the minimal plasma with the corrected concentrations of the platelets, the granulocytes, the RBCs, etc., to the subject.

The blood analysis system 360′ includes a controlled gating mechanism to flow the plasma from the centrifugation unit 102 either to the channel 179 or to the channel 181. The sequence of flow of the plasma from the channel 179 is described in detail earlier in the description with respect to FIGS. 1 to 6D.

The blood analysis system 360′ includes a detection unit 702 in the channel 181. In an example, the blood analysis system 360′ may include a holding unit 104′ in the channel 181 and placed before the detection unit 702. In the holding unit 104′, the plasma may be diluted with water, if necessary, to adjust viscosity of the plasma such that suitable flow of the plasma is achieved facilitating maximum detection, separation, and/or filtration, as the case may be, at subsequent stages of the blood analysis system 360′. The detection unit 702 receives the plasma with remaining number of the first capturing molecules and unbound molecules and ions, but without the cellular components. The detection unit 702 is operated to detect, and determine concentration of, one or more types of molecules and/or ions in the received plasma. The detection unit 702 is similar to any of the detection units described earlier in the description.

The blood analysis system 360′ further includes a correction unit 704 coupled to the detection unit 702 and placed in channel 710 from the detection unit 702. The configuration and operation of the correction unit are described earlier with reference to FIG. 3. The correction unit 704 is operated to either pump in extra one or more types of molecules and/or ions if the concentration of any is determined to be less than permissible concentration or extract out the extra amount of one or more types of molecules and/or ions if the concentration of any is determined to be more than the permissible concentration.

The correction unit 702, as explained earlier, has a two cell arrangement, wherein each cell has an SPM of a specific molecular weight cut-off that at least allows the plasma with the one or more types of molecules and/or ions to pass through. The SPMs of both the cells have the same specific molecular weight cut-off. As described earlier, one of the cells of the correction unit 702 is operated to correct the determined concentration of the one or more types of molecules and/or ions, and the other of the cells is operated for declogging.

Further, in response to determining the concentration of the one or more types of molecules and/or ions being more than permissible concentration, the cell, being operated to correct the determined concentration, is operated to: receive, in a chamber on a retentate side of its SPM, second capturing molecules having concentration depending on an excess amount of the one or more types of molecules and/or ions that are to be captured and removed from the plasma for correcting the determined concentration, where the second capturing molecules have molecular weight more than the specific molecular weight cut-off of the first and second SPM and bind with the one or more types of molecules and/or ions to make each bound molecule or ion unfunctional; remove the first capturing molecules and the second capturing molecules from the chamber on the retentate side of its SPM; and cause the one or more types of molecules and/or ions, which are not bound to the second capturing molecules, along with other molecules and/or ions to pass through its SPM to a filtrate side of the chamber. The amount of entities bound with the first capturing molecules, which are discarded, are, for example, either added back to the plasma in the collecting channel 180 before infusing the plasma back to the subject or administered to the subject independently and not from the blood analysis system.

Further, in response to determining the concentration of the one or more types of molecules and/or ions being less than permissible concentration, the cell, being operated to correct the determined concentration, is operated to: receive, in a chamber on a retentate side of its SPM, deficient amount of the one or more types of molecules and/or ions; and cause the one or more types of molecules and/or ions along with other molecules and/or ions to pass through its SPM to a filtrate side of the chamber.

Further, the blood analysis system 360′ comprises a detection unit 706, in a channel coupled to chambers of filtrate sides of the SPMs of the cells of the correction unit, to detect, and determine concentration of, the one or more types of molecules and/or ions in the plasma output from the correction unit. In response to determining the concentration, in the plasma output from the correction unit, not equal to permissible concentration of the one or more types of molecules and/or ions, the blood analysis system 360′ is operated to operate the detection unit 706 to flow the plasma back to the correction unit 704 via channel 714. The correction unit 704 is further operated to recorrect the concentration of the one or more types of molecules and/or ions. In an example, in response to determining the concentration, in the plasma output from the correction unit 704, substantially equal to the permissible concentration, the blood analysis system 360′ is operated to operate the detection unit 706 to flow the plasma to the subject via the output channel 712.

In an example, the detection unit 706 is preceded by a concentration unit 708 for concentrating the plasma output from the correction unit 704.

Further, each of the chambers on a retentate side of the SPMs of both the cells are coupled to a respective concentration unit and a respective detection unit to concentrate and determine, respectively, concentration of un-captured retentate molecules and/or ions during declogging of one of the cells—and to correct the concentration of the one or more types of molecules and/or ions, based on the concentration of the un-captured retentate molecules and/or ions by the other of the first cell or the second cell.

In an example, either or both of the SPMs in the correction unit 704 filter the one or more types of molecules and/or ions with or without at least one of centrifugation or external electromagnetic field.

Further, in an example, post determining the concentration of at least one specific type of molecules and/or ions, the plasma with remaining number of the first capturing molecules and unbound molecules and ions, but without the cellular components from the channel 710 may be passed via channel 716 to one or more of the filtration units 160-1, 160-2, 160-3 for filtration of one or more specific types of molecules and/or ions. After the filtration, concentration of one or more types of molecules and/or ions may be corrected, as described earlier in the description.

### Limitations:

It may not be possible to completely filter out every molecule and/or ion. However, at a given flow rate, pressure, characteristics of SPM with or without EMF the fraction of molecules filtered out would be a function of factors mentioned above and initial concentration. During experimentation and standardization, a graph can be made with suitable values of factors mentioned above and the said graph can be used to calculate the actual concentration of molecular or ionic entities under consideration. In an example, the ion-free plasma, as the outputted by the ion separation unit, may have traces of cations and/or anions, depending on the tolerance levels of the components of the ion separation unit involved in the separation of cations and/or anions.

The blood analysis system 360 can be used in its entirety or else it can be contracted such that only a narrow range of detectable are isolated and measured/manipulated. For patients with septicemia only Corrector may be employed to remove microbial products and excess proinflammatory cytokines. The patients in Intensive care units can benefit greatly just by making use of ion separation unit. Limited modules can be used for numerous medical conditions as some of them are listed in the introduction.

### Fabrication of the Blood Analysis System 360:

Since this kind of system has never been imagined before, it has to be built from the scratch. The blood analysis system 360 may be first standardized on medium to large mammals. Alternatively, expired human blood from blood banks or human plasma can be used.

One of the mandates of the blood analysis system 360 is to measure the detectables in real time using non-chemical methods. Whatever data is obtained using such methods has to be compared with some Gold standards. Therefore, the first step would be to take a blood volume with known concentrations of as many molecules and ionic entities as possible. The volume required via chemical methods would be appreciable if one were to ensure consistency and true value. It has to be kept in mind that even with the chemical methods depending on the equipment, methods, kits or personnel, the variation of up to 10% is not unusual even if identical sample is used. The variation appears to be unavoidable but acceptable enough to base treatment of patients in spite of it.

Second step would be to ensure the readings of non-chemical methods employed in the blood analysis system 360 and compare the results with the chemical methods/NMR, note down the differences, if any, and ascertain if the differences have a predictable pattern or are random or chaotic. The data gleaned from chemical/NMR and non-chemical methods have to be compared at each step of the system without exception. Since the rate of filtration and proportion of total molecules filtered is determined by variables such as SPM pore size, SPM pore density, SPM surface area, Rate of flow of solution in a cell C, Pressure in chambers C1 and C2 of the cell C, Number of types of molecules/unit volume, Number of each type of molecules with unique MW/unit volume, Number of anions with different valences/unit volume, Number of cations with different valences/unit volume, and Viscosity (V) of fluid entering various units of the blood analysis system 360.

During calibration all the above or some of them may have to be used with different properties (in case of SPMs) or values (in case of concentrations and pressures) till a useful reproducible pattern emerges. The process has to be repeated till statistically relevant results are obtained and reproducible algorithms are delineated.

### Applications/Uses:

The applications of the blood analysis system 360 are diverse and will expand with time. Few of the experimental and therapeutic uses are as follows:

### Experimental Uses

1. Accurate, real time and continuous measurement of all molecular/ionic entities in blood without the use of any chemical reactions will be helpful to study the trends in normal growth and aging of all mammals including humans.
2. It will elucidate trends of all disease processes as reflected in the composition of blood. It will clarify the demarcation of the state of health from the state of disease by studying these trends in all mammals.
3. It will help understand and define the process of dying. It will help mark the beginning of a state of irreversibility during dying and will possibly indicate ways to shift/change the point of irreversibility in critically ill patients.
4. It will help to describe with greater precision the changes induced in the body during iatrogenic interventions like use of drugs, use of anesthetic agents, hypothermia, hyperthermia, etc.
5. It will contribute greatly to our understanding of ‘intraoperative physiology’ with detailed and precise information of fluctuations of all molecular/ionic parameters in blood during anesthesia and surgery. Such data may potentially alter the definition of ‘high risk patient’.

### Therapeutic Uses:

### a) Uses of Chemical Capture:

1. DKA/HSS: Removal of excess glucose with titration of all other molecular/ionic entities in blood.
2. Bites, Venoms, Stings, Drug overdoses, Accidental/Intentional poisoning, Toxin mediated injury: Removal of unwanted entity from blood without subjecting the patient to any drug or chelator.
3. Electrolyte imbalance: Titration of electrolytes to precise levels without the use of drugs, resins etc.
4. Renal impairment or renal failure/Azotemia or Uremia: Titration of all molecular/ionic entities like creatinine, urea, potassium etc. to precise levels without the use of anticoagulants and without drawing a significant about of blood from the body at any given point of time thereby not subjecting the patient to iatrogenic hypotension.
5. Kernicterus due to unconjugated hyperbilirubinemia: Removal of bilirubin
6. Metabolic encephalopaties such as hepatic, renal/uremic, pulmonary encephalopathy: Titration of the undesired molecular/ionic entity
7. Acute autoimmune conditions like Hemolytic uremic syndrome, Guillian-barre syndrome etc: Complete removal of the implicated antibodies.
8. Acute flare ups of all autoimmune conditicated antibodiesions like Rheumatoid arthritis, SLE, Acute rheumatic fever etc.: Complete removal of implicated antibodies.
9. Pancreatitis and it's complications: Prevention of pancreatitis in high risk patients by removal of offending agents like implicated drugs, triglycerides, infectious agents etc. Prevention of complications of pancreatitis like ARDS by removal of leaked/spilled over proteases from blood.
10. Endocrine crises such as Thyroid storm, Pheochromocytoma, SIADH etc.: Titration of the implicated hormone.
11. Diseases with dysregulation/compensatory overdrive of the Renin Angiotensie aldosterone system (RAAS) like Acute decompensated heart faliure, Hypertensive emergency of any etiology, Hypotensive crisis: Titration of the RAAS along with titration of Vasopressin, Atrial and Brain natriuretic peptide etc.
12. Metabolic disorders of dysregulated excretion like Hemochromatosis, Wilson's disease etc.: Removal of implicated agent.
13. Infectious condition including all bacterial, fungal and viral infections including HIV/AIIDS: Complete removal from all bacterial, fungal and viral particles from blood thereby largely precluding the need for antimicrobials.
14. It will be a tool to maintain ‘intraoperative’ and ‘intra-ICU’ steady state with continuous, precise measurement and prompt titration of all molecular/ionic entities in blood.

### B) Uses of Selective Infusion and Capture at Different Sites:

1. Fluoroscopic infusion of thrombolytics such as tissue plasminogen activators into the coronary arteries following myocardial infarction with prompt extraction of the thrombolytic from the carotid arteries to reduce the risk of intracranial hemorrhage. This will markedly reduce exclusion criteria of thrombolytic interventions.
2. Selective infusion of chemotheraputic agents through the arterial end of the intended site of action with extraction from the venous end thereby negating the inadvertent adverse effects of such drugs at all other sites.
3. Removal of nephrotoxic drugs from the renal artery and hepatotoxic drugs from the hepatic artery and portal vein thereby allowing such drugs to be used in adequate doses in patients with compromised kidneys and livers respectively.

A system would be desirable and of significant value that can detect concentration of all the detectables in blood in real time without patient/subjects/animals having to lose any blood in the process. The system would also make it possible to either introduce or extract desired detectable for therapeutic and/or experimental purposes, latter in animal models.

Blood is highly complex fluid containing cellular, molecular, and ionic entities therefore in order to achieve the objectives listed above it is imperative that all the constituents are separated or isolated completely or as much as pragmatically possible. The isolation of one type of molecule without chemical reaction from a complex solution is extremely common indeed. Desalination of sea water and reverse osmosis in domestic use for water purification allow only water molecules to pass through whereas rest of the molecules are not. Dialysis on the other hand allow only a few molecules to pass through. All these processes make use of semi-permeable membranes with properties relevant to the situation at hand.

The blood analysis system 360 requires sophisticated control systems, software and extreme precision. The system probably would not have been possible even in the early 21th century but now with significant advances in nanotechnology, precision and computing power it should not be difficult to manufacture the same. The blood analysis system 360 can serve the purpose of not only understanding normal mammalian physiology but also morbid states as reflected in blood. The therapeutic applications are numerous since the blood analysis system 360 is capable of extracting molecular or ionic entities from blood without any drug.

The following description describes an example procedure for analysis of blood for detecting and correcting glucose levels using the blood analysis system 360 as described through FIG. 1. The sequence of steps for monitoring and management of a patient (i.e., a subject) of diabetes mellitus with the blood glucose level reaching beyond 600 mg/dl and causing an electrolyte disturbance (abnormal sodium, potassium levels). Conventionally, such patients are managed by drugs, such as insulin, metformin, etc., where the dose of these drugs is adjusted by repeated blood testing of parameters that are to be corrected. Molecular weight of glucose being 180. A molecular weight cut off (MWCO) of an SPM is lowest molecular weight of the solute of which 90% is retained. For instance, an SPM with the MWCO of 180 will retain 90% of the glucose and molecules larger than glucose.
Step 1: The blood of the patient is passed to centrifugation unit 102 via a channel. The channel is made of an inert material similar to what is used in other extracorporeal systems, like dialysis or plasmapheresis. Example materials include, but are not limited to, biocompatible polyvinylchloride or other material.
Step 2: The first capturing molecules for chemical capture of calcium ion and/or one or more of the coagulation molecules/ions along with complement factors are added into the centrifugation unit 102 prior to the blood entering the centrifugation unit 102. After this, the centrifugation unit 102, with or without incorporated SPM, is operated to centrifuge such that the cellular components with minimal plasma and capturing molecules are separated from majority of plasma with first capturing molecules.
Step 3: The cellular components (with or without dilution by physiological fluid) are directed towards a correction unit (not shown in FIG. 1) coupled to the centrifugation unit 102. This correction unit receives the minimal plasma having the first capturing molecules and the cellular components from the centrifugation unit 102, which extracts all the first capturing molecules from the cellular components and rest of the minimal plasma.
Step 4: The minimal plasma having the separated cellular components is infused back to the patient along with replaced captured molecules and/or ions, and the first capturing molecules are discarded.
Step 5: The separated plasma from step 2 is directed to the holding unit 104, where the plasma is diluted with water, if necessary, to control the viscosity of the plasma.
Step 6: The plasma from the holding unit 104 is directed to the ion separation unit 106, where the anions and the cations in the plasma are separated. The separation of the cations, like sodium and potassium, is effected by making use of electrodialysis, where negatively charged SPM 144, 151 of the cell 142, 143, with MWCO of 50, with/without centrifugation, with/without suitable electromagnetic field, is used so that only positively charged ions (cations) are able to pass through. The filtered cations are directed to a concentration unit 208, a detection unit 186, and then to the correction unit 130 for correction. Post correction, the cations are directed to the holding unit 156, which are then released to the collecting channel 180. The separation of anions, like chloride, etc., is effected by making use of electrodialysis, where positively charged SPM 125, 150 of the cell 110, 116, with MWCO of 50, with/without centrifugation, with/without suitable electromagnetic field, is used so that only negatively charged ions (anions) are able to pass through. The filtered anions are directed to a concentration unit 208, a detection unit 186, and then to the correction unit 130 for correction. Post correction, the anions are directed to the holding unit 156, which are then released to the collecting channel 180.
Step 7: The ion free plasma, with the first capturing molecules, from the ion separation unit 106 is directed to the filtration unit 160-1, where the SPM with MWCO of 500, with/without centrifugation, with/without suitable electromagnetic field, is used such that the entities with the molecular weight of more than 500 are retained in X1 of the filtration unit 160-1 and the entities with the molecular weight of less than 500 (including glucose with MW of 180) are filtered to Y1 of the filtration unit 160-1. The retained entities in X1 is directed to the correction unit 170-1 for correction or directly to the holding unit 172-1 for further transfer to the collecting channel 180.
Step 8a: The filtrate in Y1 of the filtration unit 160-1 (molecular weight less than 500) is directed to a concentration unit (not shown), followed by a detection unit (not shown), and a correction unit (not shown), where high levels of glucose are corrected. The corrected fluid is directed to a holding unit (not shown) and finally to the collecting channel 180.
Step 8b (optional): The filtrate in Y1 (the molecular weight less than 500) is channeled directly to next unit filtration unit 160-2, via the concentration unit 256 and the detection unit 202. In the filtration unit 160-2, each of the cells has a SPM with the MWCO of 300 and with/without centrifugation, with/without suitable electromagnetic field, the filtration unit 160-2 is operated such that the entities with molecular weight of more than 300 are retained in X2 and the entities of molecular weight less than 300 are channeled from Y2 to a concentration unit (not shown) followed by a detection unit (not shown). Then the filtrate is channeled to a correction unit (not shown) where high levels of glucose are corrected. The corrected fluid is directed to a holding unit (not shown) and finally to the collecting channel 180. Meanwhile, the retained entities in X2 are directed to a concentration unit 216, a detection unit 194, and the correction unit 170-2 and then to the holding unit 172-2, or directly to the holding unit 172-2 bypassing the concentration unit 216, the detection unit 194, and the correction unit 170-2. From the holding unit 172-2, the entities are released into the collecting channel 180.
Step 8c (optional): The entities in Y2 (the molecular weight less than 300) is directed to the next filtration unit 160-3, via the concentration unit 258 and the detection unit 204, where the SPM with MWCO of 200 is used, and with/without centrifugation, with/without suitable electromagnetic field, the entities with molecular weight of more than 200 are retained in X3 and the entities with molecular weight less than 200 from Y3 are channeled to a concentration unit (not shown) followed by a detection unit (not shown), where the concentration of glucose determined and then the filtrate is channeled to a correction unit (not shown) where high levels of glucose are corrected. The corrected fluid is directed to a holding unit (not shown) and finally to the collecting channel 180. Meanwhile, the retained entities in X3 are directed to a holding unit (not shown) and finally to the collecting channel 180.

The purpose of sequential filtration units with the SPMs of different MWCOs is to reduce the types of molecules in a given solution so that both the detection and the correction are specific and reliable. It may be possible to get the desirable results after just one filtration unit or none at all.

In an example, the blood analysis system 360 comprises a control unit (not shown) to cut-off or cause a flow of plasma and/or cellular component at each channel of the blood analysis system 360 via flow rate pumps (not shown) so as to ensure coordination between all units and eventual reconstitution of plasma before reinfusion back to the subject.

In an example, any channel as shown and described herein is made of an inert material similar to what is used in other extracorporeal systems, like dialysis or plasmapheresis. Example materials include, but are not limited to, biocompatible polyvinylchloride or other material.

### First Set of Example Embodiments

In an example, the blood analysis system 360 comprises a centrifugation unit 102 to receive blood of a subject. The centrifugation unit 102 is to hold first capturing molecules, where one first capturing molecule binds with, to chemically capture, one molecule or ion that causes coagulation of plasma; and centrifuge to suspend cellular components with a minimal plasma along with a number of the first capturing molecules. The blood analysis system 360 comprises: a holding unit 104 coupled to the centrifugation unit 102 to receive plasma with the capturing molecules but without the cellular components from the centrifugation unit 102 and hold the received plasma; and an ion separation unit 106 coupled to the holding unit 104 to receive the plasma from the holding unit 104. The ion separation unit 106 is to separate anions and cations from the received plasma based on electrostatically-charged semi permeable membranes. The blood analysis system 360 further comprises one or more filtration units 160-1, 160-2, 160-3 to receive ion-free plasma with the capturing molecules expelled from the ion separation unit 106 to filter out one or more specific types of molecules from the ion-free plasma for detection and correction, before moving the ion-free plasma having the corrected filtered molecules towards a collecting channel 180 for infusing back to the subject.

In an example, the received plasma is diluted with water in the holding unit 104 to control the viscosity of the received plasma.

In an example, the ion separation unit 106 is to separate the anions before the cations, or separate the cations before the anions.

In an example, the ion separation unit 106 is to separate the anions and the cations from the received plasma with or without at least one of centrifugation and external electromagnetic field.

In an example, the ion separation unit 106 is coupled to the holding unit 104 through a channel 108 to receive the received plasma, and the ion separation unit 106 comprises an anion separation unit and a cation separation unit.

In an example, the anion separation unit comprises a first cell 110 coupled to the channel 108 and having a first semi-permeable membrane (SPM) 125 of a specific molecular weight cut-off that allows the anions to pass through; and a second cell 116 coupled to the channel 108 and having a second SPM 150. The second SPM 150 is the same as the first SPM 125. One of the first cell 110 and the second cell 116 is operated to separate the anions and the other of the first cell 110 and the second cell 116 is operated for declogging. The filtered anions are passed from channels 126 or 123 to channel 131 and then to the output channel 224. The output channel 224 is connected to a concentration unit 208 (optionally) and to a detection unit 186 which is coupled to a correction unit 130 to correct a concentration of anions expelled from the detection unit 184 before passing the corrected anions towards the collecting channel 180.

In an example, the cation separation unit comprises a third cell 142 coupled to a channel 129 to receive a retentate plasma from the first cell 110 and the second cell 116 and having a third semi-permeable membrane (SPM) 144 of a specific molecular weight cut-off that allows the cations to pass through; and a fourth cell 143 coupled to the channel 129 to receive a retentate plasma from the first cell 110 and the second cell 116 and having a fourth SPM 151. The fourth SPM 151 is the same as the third SPM 144. One of the third cell 142 and the fourth cell 143 is operated to separate the cations and the other of the third cell 142 and the fourth cell 143 is operated for declogging. The filtered cations are passed from channels 140 or 137 to channel 149 and then to the output channel 224. The output channel 224 is connected to a concentration unit 208 (optionally) and to a detection unit 186 which is coupled to a correction unit 130 to correct a concentration of cations expelled from the detection unit 184 before passing the corrected cations towards the collecting channel 180.

In an example, the first SPM 125 and the second SPM 150, are positively charged and the third SPM 144 and the fourth SPM 151 are negatively charged, when the anion separation unit is positioned before the cation separation unit in the ion separation unit 106.

In an example, the first SPM 125 and the second SPM 150, are negatively charged and the third SPM 144 and the fourth SPM 151 are positively charged, when the cation separation unit is positioned before the anion separation unit in the ion separation unit 106.

In an example, each filtration unit 160-1 comprises a fifth cell 446 coupled to a channel 400 to receive the ion-free plasma from the ion separation unit 106 and having a fifth SPM 448 of a specific molecular weight cut-off that allows molecules of a molecular weight equal to or less than the specific molecular weight cut-off to pass through; and a sixth cell 450 coupled to the channel 400 to receive the ion-free plasma from the ion separation unit 106 and having a sixth SPM 452. The sixth SPM 45 being the same as the fifth SPM 446. One of the fifth cell 446 and the sixth cell 450 is operated to filter the molecules and the other of the fifth cell 446 and the sixth cell 450 is operated for declogging. The molecules from the retentate side of the fifth and sixth SPMs 448, 452 of the corresponding chambers are passed from channels 426 and 428 to the output channel 434 which is coupled to a correction unit 170-1, 170-2, 170-3 to correct a concentration of molecules determined by the detection unit 190, 194, 196. The corrected concentration of molecules is again determined by a further detection unit 192, 196, 200 based on which the concentration of the molecules may be re-corrected before passing the corrected molecules towards the collecting channel 180. The molecules from the filtrate side of the fifth and sixth SPMs 448, 452 of the corresponding chambers are passed from channels 436 and 438 to the output channel 444 from which the molecules may be passed to a subsequent filtration unit 160-2, 160-3, via a concentration unit 256, 258 and a detection unit 202, 204, for sequential filtration of molecules of a specific molecular weight less than the specific molecular weight cut-off associated with a previous filtration unit 160-1 or passed to the collecting channel.

In an example, each of the correction units 130, 170-1, 170-2, 170-3 comprises a seventh cell 340 coupled to a channel 300 to receive plasma with the first capturing molecules and with molecules and with or without ions that are to be corrected and having a seventh SPM 344 of a specific molecular weight cut-off that allows molecules and/or ions of a molecular weight equal to or less than the specific molecular weight cut-off to pass through; and an eighth cell 342 coupled to the channel 300 to receive plasma with the first capturing molecules and with molecules and with or without ions that are to be corrected and having an eighth SPM 346. The eighth SPM 346 is the same as the seventh SPM 344. One of the seventh cell 340 and the eighth cell 342 is operated to separate the molecules and/or ions and/or the first capturing molecules and the other of the seventh cell 340 and the eighth cell 342 is operated for declogging. The first capturing molecules are removed from one of the seventh cell 340 and eighth cell 342 and discarded and the amount of molecules equal to those bound with the first capturing molecules and discarded are added for example in the collection channel 180 before infusing the plasma to the subject. Chambers on a retentate side of the seventh and eighth SPMs 344, 346 of each of the seventh cell 340 and the eighth cell 342 are coupled to a channel 320, 318 to receive second capturing molecules having a concentration depending on an amount of excess molecules and/or ions that are to be captured and removed from the plasma via the chambers on the retentate side or to receive an amount of molecules and/or ions bring the concentration of the molecules and/or ions up to the permissible concentration. The chambers on the retentate side of the seventh and eighth SPMs 344, 346 of each of the seventh cell 340 and the eighth cell 342 are coupled to a respective concentration unit 354, 358 and a respective detection unit 356, 360 to concentrate and detect, respectively, un-captured retentate molecules and/or ions during declogging of the seventh cell 340 or the eighth cell 342. The concentration of the amount of deficient molecules and/or ions that is added through the channel 320, 318 during declogging of the seventh cell 340 or the eight cell 342. Chambers on a filtrate side of the seventh and eighth SPMs 344, 346 of each of the seventh cell 340 and the eighth cell 342 are respective coupled to channel 314 and 324 which are further coupled to the output channel 326. The output channel 326 which is coupled to a correction unit 130, 170-1, 170-2, 170-3 to correct concentration of molecules and/or ions determined by the detection unit 186, 190, 194, 196. The corrected concentration of molecules and/or ions is again determined by a further detection unit 188, 192, 196, 200 based on which the concentration of the molecules and/or ions can be re-corrected before passing the corrected molecules and/or ions towards the collecting channel 180.

In an example, each concentration unit comprises a ninth cell 506 with a symmetrical arrangement of SPMs 508, 510 and a channel 504 to receive plasma in a region between the symmetrical arrangement of SPMs 508, 510; a tenth cell 526 with a symmetrical arrangement of SPMs 530, 532 and a channel 524 to receive plasma in a region between the symmetrical arrangement of SPMs 530, 532; and an arrangement to allow water to circulate through the ninth cell 506 and the tenth cell 526, where one of the ninth cell 506 and the tenth cell 526 is operated to increase a concentration of the plasma and the other of the ninth cell 506 and the tenth cell 526 is operated for declogging, where the water is circulated to create an osmotic pressure in the regions between the symmetrical arrangement of SPMs 508, 510, 530, 532 and confine concentrated plasma to one of an area 518 and an area 536 that is being operated for concentration of the plasma.

In an example, each detection unit comprises a plurality of pairs of bifurcation channels 604, 606, 608, 610, 612, 614, . . . , where each pair of bifurcation channels 606, 608 is to bifurcate plasma from a previous bifurcation channel 604 or from an inlet channel 602, through which plasma for detection of molecules is received, into two substantially equal plasma streams; at least one of a spectroscopy unit and a chemical reaction-based measurement unit installed in each bifurcation channel to determine a concentration of the molecules and/or the ions in the respective bifurcation channel; and a plurality of converging channels 632, 634, 636, 638, . . . , where each converging channel is to combine two plasma streams from a pair of bifurcation channels or from two converging channels into a single stream.

In an example, the detection unit is to determine whether the detected concentration of the molecules and/or the ions is less than or more than a predefined value; in response to detecting that the concentration is less than the predefined value, add a specific amount of the molecules and/or the ions to match the predefined value; and in response to detecting that the concentration is more than the predefined value, provide information to the correction unit about an amount of the molecules and/or the ions that are in excess, where the correction unit is to operate the channel 318, 320 to provide a specific amount to capturing molecules into one of the seventh cell 340 and eighth cell 342 to remove the excess amount of the molecules and/or the ions.

In an example, the blood analysis system 360 comprising a correction unit coupled to the centrifugation unit 102 to receive the minimal plasma having the first capturing molecules and the cellular components from the centrifugation unit 102, where the correction unit is to extract the first capturing molecules from the minimal plasma.

In an example, the minimal plasma having the cellular components is infused back to the subject along with replaced captured molecules and/or ions that were bound with the first capturing molecules, and the extracted first capturing molecules are discarded.

In an example, the minimal plasma having the cellular components are suspended in an isotonic solution, and optionally with oxygen and glucose, before infusing back of the subject.

In an example, the blood analysis system 360 comprises an SPM and/or a centrifuge to separate platelets from the minimal plasma; a detection unit to receive the separate platelets and count the platelets; and a correction unit to correct the count of the platelets before infusing the minimal plasma with the corrected platelets to the subject.

In an example, the minimal plasma, after separating the platelets, is ultra-centrifuged to separate granulocytes from the minimal plasma, where the blood analysis system 360 comprises a detection unit to count the granulocytes; and a correction unit to correct the count of the granulocytes, before infusing the minimal plasma with the corrected granulocytes to the subject.

In an example, the minimal plasma, after separating the platelets, is ultra-centrifuged to separate red blood cells (RBCs) from the minimal plasma, where the blood analysis system 360 comprises a detection unit to count the RBCs; and a correction unit to correct the count of the RBCs, before infusing the minimal plasma with the corrected RBCs to the subject.

In an example, molecules and/or ions, equivalent to the molecules and/or the ions captured by the discarded capturing molecules, are added before infusing the plasma back to the subject.

In an example, the blood analysis system 360 comprises a control unit to cut-off or cause a flow of plasma and/or cellular component at each channel of the blood analysis system 360 via flow rate pumps.

### Second Set of Example Embodiments

In an example, the blood analysis system 360 comprises a centrifugation unit 102 to receive blood of a subject. The centrifugation unit 102 is to hold first capturing molecules, where one first capturing molecule binds with, to chemically capture, one molecule or ion that causes coagulation of plasma; and centrifuge to suspend cellular components with a minimal plasma along with a number of the first capturing molecules. The blood analysis system 360 comprises a correction unit coupled to the centrifugation unit 102 to receive the minimal plasma having the first capturing molecules and the cellular components from the centrifugation unit 102, where the correction unit is to extract the first capturing molecules from the minimal plasma, prior to infusing the minimal plasma having the cellular components along with replaced captured molecules and/or ions back to the subject and discarding the extracted capturing molecules.

In an example, the minimal plasma having the cellular components are suspended in an isotonic solution, and optionally with oxygen and glucose, before infusing back of the subject.

In an example, the blood analysis system 360 comprises an SPM and/or a centrifuge to separate platelets from the minimal plasma; a detection unit to receive the separate platelets and count the platelets; and a correction unit to correct the count of the platelets before infusing the minimal plasma with the corrected platelets to the subject.

In an example, the minimal plasma, after separating the platelets, is ultracentrifuged to separate granulocytes from the minimal plasma, and where the blood analysis system 360 comprises: a detection unit to count the granulocytes; and a correction unit to correct the count of the granulocytes, before infusing the minimal plasma with the corrected granulocytes to the subject.

In an example, the minimal plasma, after separating the platelets, is ultracentrifuged to separate red blood cells (RBCs) from the minimal plasma, and where the blood analysis system 360 comprises: a detection unit to count the RBCs; and a correction unit to correct the count of the RBCs, before infusing the minimal plasma with the corrected RBCs to the subject.

In an example, the blood analysis system 360 comprises: a holding unit 104 coupled to the centrifugation unit 102 to receive plasma with the remaining first capturing molecules and other molecules and ions but without the cellular components from the centrifugation unit 102 and hold the received plasma; one or more filtration units 160-1, 160-2, 160-3 to receive the plasma with the first capturing molecules and other molecules and ions but without the cellular components expelled from the holding unit 104 to filter out one or more specific types of molecules from the plasma for detection and correction, before moving the plasma having the corrected filtered molecules towards a collecting channel 180 for infusing back to the subject. Other functional operations performed by a filtration unit 160 before infusing the plasma back to the subject are the same as described earlier for the first set of example embodiments.

In an example, the received plasma is diluted with water in the holding unit 104 to control the viscosity of the received plasma.

In an example, the blood analysis system 360 comprises an ion separation unit 106 between the holding unit 104 and the at least one filtration unit 160-1, 160-2, 160-3 to receive the plasma from the holding unit 104, where the ion separation unit 106 is to separate anions and cations from the received plasma based on electrostatically-charged semi permeable membranes.

In an example, the ion separation unit 106 is to: separate the anions before the cations, or separate the cations before the anions.

In an example, the ion separation unit 106 is to separate the anions and the cations from the received plasma with or without at least one of centrifugation and external electromagnetic field.

In an example, the ion separation unit 106 is coupled to the holding unit 104 through a channel 108 to receive the plasma, and the ion separation unit 106 comprises an anion separation unit and a cation separation unit.

In an example, the anion separation unit comprises: a first cell 110 coupled to the channel 108 and having a first semi-permeable membrane (SPM) 125 of a specific molecular weight cut-off that allows the anions to pass through; and a second cell 116 coupled to the channel 108 and having a second SPM 150, where the second SPM 150 is the same as the first SPM 125. One of the first cell 110 and the second cell 116 is operated to separate the anions and the other of the first cell 110 and the second cell 116 is operated for declogging. The filtered anions are passed from channels 126 or 123 to channel 131 and then to the output channel 224. The output channel 224 is connected to a concentration unit 208 (optionally) and to a detection unit 186 which is coupled to a correction unit 130 to correct a concentration of anions expelled from the detection unit 184 before passing the corrected anions towards the collecting channel 180.

In an example, the cation separation unit comprises: a third cell 142 coupled to a channel 129 to receive a retentate plasma from the first cell 110 and the second cell 116 and having a third semi-permeable membrane (SPM) 144 of a specific molecular weight cut-off that allows the cations to pass through; and a fourth cell 143 coupled to the channel 129 to receive a retentate plasma from the first cell 110 and the second cell 116 and having a fourth SPM 151, where the fourth SPM 151 is the same as the third SPM 144. One of the third cell 142 and the fourth cell 143 is operated to separate the cations and the other of the third cell 142 and the fourth cell 143 is operated for declogging. The filtered cations are passed from channels 140 or 137 to channel 149 and then to the output channel 224. The output channel 224 is connected to a concentration unit 208 (optionally) and to a detection unit 186 which is coupled to a correction unit 130 to correct a concentration of cations expelled from the detection unit 184 before passing the corrected cations towards the collecting channel 180.

In an example, the first SPM 125 and the second SPM 150, are positively charged and the third SPM 144 and the fourth SPM 151 are negatively charged, when the anion separation unit is positioned before the cation separation unit in the ion separation unit 106.

In an example, the first SPM 125 and the second SPM 150, are negatively charged and the third SPM 144 and the fourth SPM 151 are positively charged, when the cation separation unit is positioned before the anion separation unit in the ion separation unit 106.

In an example, each filtration unit 160-1 comprises: a fifth cell 446 coupled to a channel 400 to receive the plasma and having a fifth SPM 448 of a specific molecular weight cut-off that allows molecules and/or ions, as the case may be, of a molecular weight equal to or less than the specific molecular weight cut-off to pass through; and a sixth cell 450 coupled to the channel 400 to receive the plasma and having a sixth SPM 452, where the sixth SPM 452 being the same as the fifth SPM 446, where one of the fifth cell 446 and the sixth cell 450 is operated to filter the molecules and the other of the fifth cell 446 and the sixth cell 450 is operated for declogging. The molecules and/ions from the retentate side of the fifth and sixth SPMs 448, 452 of the corresponding chambers are passed from channels 426 and 428 to the output channel 434 which is coupled to a correction unit 170-1, 170-2, 170-3 to correct a concentration of molecules and/or ions determined by the detection unit 190, 194, 196. The corrected concentration of molecules and/or ions is again determined by a further detection unit 192, 196, 200 based on which the concentration of the molecules and/or ions may be re-corrected before passing the corrected molecules towards the collecting channel 180. The molecules from the filtrate side of the fifth and sixth SPMs 448, 452 of the corresponding chambers are passed from channels 436 and 438 to the output channel 444 from which the molecules may be passed to a subsequent filtration unit 160-2, 160-3, via a concentration unit 256, 258 and a detection unit 202, 204, for sequential filtration of molecules of a specific molecular weight less than the specific molecular weight cut-off associated with a previous filtration unit 160-1 or passed to the collecting channel.

In an example, each of the correction units 130, 132, 170-1, 170-2, 170-3 comprises: a seventh cell 340 coupled to a channel 300 to receive plasma with the first capturing molecules and with molecules and with or without ions that are to be corrected and having a seventh SPM 344 of a specific molecular weight cut-off that allows molecules and/or ions of a molecular weight equal to or less than the specific molecular weight cut-off to pass through; and an eighth cell 342 coupled to the channel 300 to receive plasma with the capturing molecules and with molecules and with or without ions that are to be corrected and having an eighth SPM 346, where the eighth SPM 346 is the same as the seventh SPM 344, where one of the seventh cell 340 and the eighth cell 342 is operated to separate the molecules and/or ions and/or the first capturing molecules and the other of the seventh cell 340 and the eighth cell 342 is operated for declogging. The first capturing molecules are removed from one of the seventh cell 340 and eighth cell 342 and discarded and the amount of molecules equal to those bound with the first capturing molecules and discarded are added for example in the collection channel 180 before infusing the plasma to the subject. Chambers on a retentate side of the seventh and eighth SPMs 344, 346 of each of the seventh cell 340 and the eighth cell 342 are coupled to a channel 320, 318 to receive second capturing molecules having a concentration depending on an amount of excess molecules and/or ions that are to be captured and removed from the plasma via the chambers on the retentate side or to receive an amount of molecules and/or ions bring the concentration of the molecules and/or ions up to the permissible concentration. The chambers on the retentate side of the seventh and eighth SPMs 344, 346 of each of the seventh cell 340 and the eighth cell 342 are coupled to a respective concentration unit 354, 358 and a respective detection unit 356, 360 to concentrate and detect, respectively, un-captured retentate molecules and/or ions during declogging of the seventh cell 340 or the eighth cell 342. The concentration of the amount of deficient molecules and/or ions that is added through the channel 320, 318 during declogging of the seventh cell 340 or the eight cell 342. Chambers on a filtrate side of the seventh and eighth SPMs 344, 346 of each of the seventh cell 340 and the eighth cell 342 are respective coupled to channel 314 and 324 which are further coupled to the output channel 326. The output channel 326 which is coupled to a correction unit 130, 170-1, 170-2, 170-3 to correct concentration of molecules and/or ions determined by the detection unit 186, 190, 194, 196. The corrected concentration of molecules and/or ions is again determined by a further detection unit 188, 192, 196, 200 based on which the concentration of the molecules and/or ions can be re-corrected before passing the corrected molecules and/or ions towards the collecting channel 180.

In an example, each concentration unit comprises a ninth cell 506 with a symmetrical arrangement of SPMs 508, 510 and a channel 504 to receive plasma in a region between the symmetrical arrangement of SPMs 508, 510; a tenth cell 526 with a symmetrical arrangement of SPMs 530, 532 and a channel 524 to receive plasma in a region between the symmetrical arrangement of SPMs 530, 532; and an arrangement to allow water to circulate through the ninth cell 506 and the tenth cell 526, where one of the ninth cell 506 and the tenth cell 526 is operated to increase a concentration of the plasma and the other of the ninth cell 506 and the tenth cell 526 is operated for declogging, where the water is circulated to create an osmotic pressure in the regions between the symmetrical arrangement of SPMs 508, 510, 530, 532 and confine concentrated plasma to one of an area 518 and an area 536 that is being operated for concentration of the plasma.

In an example, each detection unit comprises: a plurality of pairs of bifurcation channels 604, 606, 608, 610, 612, 614, . . . , where each pair of bifurcation channels 606, 608 is to bifurcate plasma from a previous bifurcation channel 604 or from an inlet channel 602, through which plasma for detection of molecules is received, into two substantially equal plasma streams; at least one of a spectroscopy unit and a chemical reaction-based measurement unit installed in each bifurcation channel to determine a concentration of the molecules and/or the ions in the respective bifurcation channel; and a plurality of converging channels 632, 634, 636, 638, . . . , where each converging channel is to combine two plasma streams from a pair of bifurcation channels or from two converging channels into a single stream.

In an example, the detection unit is to: determine whether the detected concentration of the molecules and/or the ions is less than or more than a predefined value; in response to detecting that the concentration is less than the predefined value, add a specific amount of the molecules and/or the ions to match the predefined value; and in response to detecting that the concentration is more than the predefined value, provide information to the correction unit about an amount of the molecules and/or the ions that are in excess, where the correction unit is to operate the channel 318, 320 to provide a specific amount to second capturing molecules into one of the seventh cell 340 or eighth cell 342 to remove the excess amount of the molecules and/or the ions.

In an example, molecules and/or ions, equivalent to the molecules and/or the ions captured by the discarded first capturing molecules, are added before infusing the plasma back to the subject.

In an example, the blood analysis system 360 comprises a control unit to cut-off or cause a flow of plasma and/or cellular component at each channel of the blood analysis system 360 via flow rate pumps.

### Third Set of Example Embodiments

In an example, the blood analysis system 360, 360′ comprises a centrifugation unit 102 to receive blood of a subject. The centrifugation unit 102 is to: hold first capturing molecules, wherein one first capturing molecule binds with, to chemically capture, one molecule or ion that causes coagulation of the plasma, wherein the binding of one first capturing molecule with a molecule or an ion renders the bound molecule or ion unfunctional; and centrifuge to suspend cellular components with a minimal plasma along with a number of the first capturing molecules. The blood analysis system 360, 360′ comprises a detection unit 702 to receive the plasma with remaining number of the first capturing molecules and unbound molecules and ions, but without the cellular components, wherein the detection unit 702 is to detect, and determine concentration of, one or more types of molecules and/or ions in the received plasma; and a correction unit 704, coupled to the detection unit 702. The correction unit 704 comprises: a first cell to receive, from the detection unit 702, the plasma and having a first semi-permeable membrane (SPM) of a specific molecular weight cut-off that at least allows the plasma with the one or more types of molecules and/or ions to pass through; and a second cell to receive, from the first detection unit 702, the plasma and having a second SPM, the second SPM being the same as the first SPM. One of the first cell or the second cell is operated to correct the determined concentration of the one or more types of molecules and/or ions, and the other of the first cell or the second cell is operated for declogging. In an example, in response to determining the concentration of the one or more types of molecules and/or ions being more than permissible concentration, the first cell or the second cell, being operated to correct the determined concentration, is operated to: receive, in a chamber on a retentate side of the first or second SPM, second capturing molecules having concentration depending on an excess amount of the one or more types of molecules and/or ions that are to be captured and removed from the plasma for correcting the determined concentration, where the second capturing molecules have molecular weight more than the specific molecular weight cut-off of the first and second SPM and bind with the one or more types of molecules and/or ions to make each bound molecule or ion unfunctional; remove the first capturing molecules and the second capturing molecules from the chamber on the retentate side; and cause the one or more types of molecules and/or ions, which are not bound to the second capturing molecules, along with other molecules and/or ions to pass through the first or second SPM to a filtrate side of the chamber. In an example, in response to determining the concentration of the one or more types of molecules and/or ions being less than permissible concentration, the first cell or the second cell, being operated to correct the determined concentration, is operated to: receive, in a chamber on a retentate side of the first or second SPM, deficient amount of the one or more types of molecules and/or ions; remove the first capturing molecules from the chamber on the retentate side; and cause the one or more types of molecules and/or ions along with other molecules and/or ions to pass through the first or second SPM to a filtrate side of the chamber.

In an example, the blood analysis system 360′ further comprises a detection unit 706, in a channel coupled to chambers of filtrate sides of the first and second SPMs of the first and second cells, to detect, and determine concentration of, the one or more types of molecules and/or ions in the plasma output from the correction unit. In response to determining the concentration, in the plasma output from the correction unit 704, not equal to permissible concentration of the one or more types of molecules and/or ions, the blood analysis system 360′ is operated to flow the plasma back to the correction unit 704, where the correction unit 704 is further operated to recorrect the concentration of the one or more types of molecules and/or ions. In an example, in response to determining the concentration, in the plasma output from the correction unit 704, substantially equal to the permissible concentration, the blood analysis system 360′ is operated to flow the plasma to the subject.

In an example, chambers on a retentate side of the first and second SPMs of each of the first cell and the second cell are coupled to a respective concentration unit and a respective detection unit to concentrate and determine, respectively, concentration of un-captured retentate molecules and/or ions during declogging of the first cell or the second cell and to correct the concentration of the one or more types of molecules and/or ions, based on the concentration of the un-captured retentate molecules and/or ions by the other of the first cell or the second cell.

In an example, the first SPM and/or the second SPM filter the one or more types of molecules and/or ions with or without at least one of centrifugation or external electromagnetic field.

In an example, the blood analysis system 360′, 360 further comprises: a detection unit 182 to receive the plasma with the remaining number of the first capturing molecules and the unbound molecules and ions, but without the cellular components, wherein the detection unit 182 is to detect, and determine concentration of, cations and/or anions in the received plasma; and an ion separation unit 106 coupled to the detection unit 182 to receive the plasma from the detection unit 182. The ion separation unit 106 is to filter ions from the received plasma based on SPMs with or without at least one of centrifugation or external electromagnetic field. The ion separation unit 106 comprises: a third cell and having a third SPM of a specific molecular weight cut-off that allows one of anions or cations to pass through; and a fourth cell and having a fourth SPM, the fourth SPM being the same as the third SPM. One of the third cell or the fourth cell is operated to filter one of the anions or cations, and the other of the third cell or the fourth cell is operated for declogging. In an example, the ion separation unit 106 comprises an anion separation unit and a cation separation unit. The ion separation unit 106 is to: separate the anions before the cations; or separate the cations before the anions. In an example, the third SPM is positively charged and the fourth SPM are negatively charged, when the anion are separated before the cations, and wherein the third SPM is negatively charged and the fourth SPM are positively charged, when the cations are separated before the anions. In an example, the blood analysis system 360′, 360 further comprises: a detection unit 186 coupled to chambers on filtrate sides of the third SPM of the third cell and the fourth SPM of the fourth cell to detect, and determine concentration of, the filtered anions and cations; and a correction unit 130 coupled to the detection unit to correct the concentration of filtered anions and/or cations expelled from the detection unit. In an example, the blood analysis system 360′, 360 further comprises a detection unit 188 to receive the corrected anions and/or cations and to detect, and determine concentration of, the corrected anions and/or cations output from the correction unit. In response to determining the concentration of the corrected cations and/or anions being not equal to a permissible concentration thereof, the blood analysis system 360′, 360 is operated to flow the cations and/or the anions the back to the correction unit 130, where the correction unit 130 is further operated to recorrect the concentration of the cations and/or the anions. In response to determining the concentration of the corrected cations and/or anions being equal to the permissible concentration thereof, the blood analysis system 360′, 360 is operated to flow the cations and/or the anions to the subject.

In an example, the blood analysis system 360′, 360 further comprises: a detection unit 184 to receive ion-free plasma with the first capturing molecules expelled from the ion separation unit 106, where the detection unit 184 is to determine concentration of one or more specific types of molecules in the received plasma; and one or more filtration units 160-1, 160-2, 160-3 coupled to the detection unit 184 to receive ion-free plasma with the first capturing molecules expelled from the ion separation unit 106. One filtration unit is to filter a specific type of molecules based on SPMs with or without at least one of centrifugation or external electromagnetic field, from the ion-free plasma. Each of the one or more filtration units 160-1, 160-2, 160-3 comprises: a fifth cell and having a fifth SPM of a specific molecular weight cut-off that allows molecules with molecular weight equal to or less than the specific molecular weight cut-off to pass through; and a sixth cell and having a sixth SPM, the sixth SPM being the same as the fifth SPM. One of the fifth cell or the sixth cell is operated to filter the specific type of molecules, and the other of the fifth cell or the sixth cell is operated for declogging. In an example, the blood analysis system 360′, 360 further comprises: a detection unit 190, 194, 198 coupled to chambers on retentate sides of the fifth SPM of the fifth cell and the sixth SPM of the sixth cell to detect, and determine concentration of, the filtered specific type of molecules expelled from the retentate side of the fifth cell or the sixth cell; and a correction unit 170-1, 170-2, 170-3 coupled to the detection unit 190, 194, 198 to correct the concentration of filtered specific type of molecules expelled from the detection unit 190, 194, 198. In an example, the blood analysis system 360′, 360 further comprises a detection unit 192, 196, 200 to receive the corrected specific type of molecules and to detect, and determine concentration of, the corrected specific type of molecules output from the correction unit 170-1, 170-2, 170-3. In response to determining the concentration of the corrected specific type of molecules being not equal to a permissible concentration thereof, the blood analysis system 360′, 360 is operated to flow the specific type of molecules the back to the correction unit, where the correction unit 170-1, 170-2, 170-3 is further operated to recorrect the concentration of the specific type of molecules. In response to determining the concentration of the corrected specific type of molecules being equal to the permissible concentration thereof, the blood analysis system 360′, 360 is operated to flow the specific type of molecules to the subject. In an example, the molecules from filtration sides of the fifth SPM of the fifth cell and the sixth SPM of the sixth cell are returned to the subject or passed to a next filtration unit of the blood analysis system.

In an example, the blood analysis system 360′, 360 further comprises one or more filtration units 160-1, 160-2, 160-3 coupled to the detection unit 702 to receive the plasma with remaining number of the first capturing molecules and unbound molecules and ions, but without the cellular components, where one filtration unit is to filter a specific type of molecules based on SPMs with or without at least one of centrifugation or external electromagnetic field, from the received plasma. Each of the one or more filtration units 160-1, 160-2, 160-3 comprises: a fifth cell and having a fifth SPM of a specific molecular weight cut-off that allows molecules with molecular weight equal to or less than the specific molecular weight cut-off to pass through; and a sixth cell and having a sixth SPM, the sixth SPM being the same as the fifth SPM. One of the fifth cell or the sixth cell is operated to filter the specific type of molecules, and the other of the fifth cell or the sixth cell is operated for declogging.

In an example, the blood analysis system 360′, 360 further comprises: a detection unit 190, 194, 198 coupled to chambers on retentate sides of the fifth SPM of the fifth cell and the sixth SPM of the sixth cell to detect, and determine concentration of, the filtered specific type of molecules expelled from the retentate side of the fifth cell or the sixth cell; and a correction unit 170-1, 170-2, 170-3 coupled to the detection unit to correct the concentration of filtered specific type of molecules expelled from the detection unit 190, 194, 198. In an example, the blood analysis system 360′, 360 further comprises a detection unit 192, 196, 200 to receive the corrected specific type of molecules and to detect, and determine concentration of, the corrected specific type of molecules output from the correction unit 170-1, 170-2, 170-3. In response to determining the concentration of the corrected specific type of molecules being not equal to a permissible concentration thereof, the blood analysis system 360′, 360 is operated to flow the specific type of molecules the back to the correction unit 170-1, 170-2, 170-3, where the correction unit 170-1, 170-2, 170-3 is further operated to recorrect the concentration of the specific type of molecules. In response to determining the concentration of the corrected specific type of molecules being equal to the permissible concentration thereof, the blood analysis system 360′, 360 is operated to flow the specific type of molecules to the subject. In an example, the molecules from filtration sides of the fifth SPM of the fifth cell and the sixth SPM of the sixth cell are returned to the subject or passed to a next filtration unit of the blood analysis system 360′, 360.

In an example, wherein the blood analysis system 360′, 360 comprises a holding unit 104. 104′ coupled to the centrifugation unit 102 to receive the plasma with the remaining number of the first capturing molecules and unbound molecules and ions, but without the cellular components from the centrifugation unit 102, where the received plasma is diluted with water in the holding unit 104, 104′ to control the viscosity of the received plasma.

In an example, the detection unit 702, 706 comprises a single channel 350 and a spectroscopy-based measurement unit, an optical sensing unit, a non-chemical sensing unit, an electrochemical sensing unit, a chemical reaction-based measurement unit, or a combination thereof, installed in the single channel 350 to determine the concentration of the one or more types of molecules and/or ions.

In an example, the detection unit 702, 706 comprises: one or more pairs of bifurcation channels 604, 606, 608, 610, 612, 614, . . . , where a pair of bifurcation channels 606, 608 is to bifurcate plasma from a previous bifurcation channel 604 or from an inlet channel 602, through which plasma for detection, and determining concentration, of the one or more types of molecules and/or ions is received, into two substantially equal plasma streams; a spectroscopy-based measurement unit, an optical sensing unit, a non-chemical sensing unit, electrochemical sensing unit, a chemical reaction-based measurement unit, or a combination thereof, installed in each bifurcation channel to determine a concentration of the one or more types of molecules and/or ions in the respective bifurcation channel; and one or more converging channels 632, 634, 636, 638, . . . , where each converging channel is to combine two plasma streams from a pair of bifurcation channels into a single plasma stream.

In an example, the blood analysis system 360′ further comprises a concentration unit 708 to receive the plasma output from the correction unit 704 and to provide the plasma to the detection unit 706. The concentration unit 708 comprises: a seventh cell with a symmetrical arrangement of SPMs and a channel to receive plasma in a region between the symmetrical arrangement of SPMs; a eighth cell with a symmetrical arrangement of SPMs and a channel to receive plasma in a region between the symmetrical arrangement of SPMs; and an arrangement to allow water to circulate through the seventh cell and the eight cell. One of the seventh cell and the eighth cell is operated to increase a concentration of the plasma and the other of the seventh cell and the eighth cell is operated for declogging, where the water is circulated to create an osmotic pressure in the regions between the symmetrical arrangement of SPMs and confine concentrated plasma to an area of the seventh cell or an area of the eighth cell that is being operated for concentration of the plasma.

In an example, the blood analysis system 360′, 360 comprises a correction unit coupled to the centrifugation unit 102 to receive the minimal plasma having the first capturing molecules and the cellular components from the centrifugation unit 102. The correction unit is to extract the first capturing molecules from the minimal plasma. In an example, the minimal plasma having the cellular components is infused back to the subject along with replaced captured molecules and/or ions, and wherein the extracted first capturing molecules are discarded. In an example, the minimal plasma having the cellular components are suspended in an isotonic solution, and optionally with oxygen and glucose, before infusing back of the subject.

In an example, the blood analysis system 360′, 360 comprises: an SPM and/or a centrifuge to separate platelets from the minimal plasma; a detection unit to receive the separate platelets and count the platelets; and a correction unit to correct the count of the platelets before infusing the minimal plasma with the corrected platelets to the subject. In an example, the minimal plasma, after separating the platelets, is ultra-centrifuged to separate granulocytes from the minimal plasma. In an example, the blood analysis system 360′, 360 comprises: a detection unit to count the granulocytes; and a correction unit to correct the count of the granulocytes, before infusing the minimal plasma with the corrected granulocytes to the subject. In an example, the minimal plasma, after separating the platelets, is ultra-centrifuged to separate red blood cells (RBCs) from the minimal plasma. In an example, the blood analysis system 360′, 360 comprises: a detection unit to count the RBCs; and a correction unit to correct the count of the RBCs, before infusing the minimal plasma with the corrected RBCs to the subject.

In an example, molecules and/or ions, equivalent to the molecules and/or the ions captured by the removed first capturing molecules, are added before infusing the plasma back to the subject.

In an example, the blood analysis system 360′, 360 comprises a control unit to cut-off or cause a flow of plasma and/or cellular component at each channel of the blood analysis system 360′, 360 via flow rate pumps.

### LIST OF CITED REFERENCES

Ref1: Anal Chem. 2002 Aug. 1; 74 (15): 420A-426A. The new wave of ion-selective electrodes. Ernö Pretsch.
Ref2: J Diabetes Sci Technol. 2008 September; 2 (5): 882-889. Real-Time Continuous Glucose Monitoring in the Clinical Setting: The Good, the Bad, and the Practical. Irene Mamkin, Svetlana Ten Sonal Bhandari, Neesha Ramchandani.
Ref3: Pulse oximetry. Jubran A. Crit Care. 2015 Jul. 16; 19:272.
Ref4: Kraft J C, Osterhaus G L, Ortiz A N, Garris P A, Johnson M A (2009) In vivo dopamine release and uptake impairments in rats treated with 3-nitropropionic acid. Neuroscience 161 (3): 940-949.
Ref5: Zhang J, et al. (2013) In vivo monitoring of serotonin in the striatum of freely moving rats with one minute temporal resolution by online microdialysis-capillary high-performance liquid chromatography at elevated temperature and pressure. Anal Chem 85 (20): 9889-9897.
Ref6: Wassum K M, et al. (2012) Transient extracellular glutamate events in the basolateral amygdala track reward-seeking actions. J Neurosci 32 (8): 2734-2746.
Ref7: Sarter M, Kim Y (2015) Interpreting chemical neurotransmission in vivo: techniques, time scales, and theories. ACS Chem Neurosci 6 (1): 8-10.
Ref8: Couture M, Zhao S S, Masson J-F (2013) Modern surface plasmon resonance for bioanalytics and biophysics. Phys Chem Chem Phys 15 (27): 11190-11216.
Ref9: Thompson M, Sheikh S, Blaszykowski C (2014) A perspective on the application of biosensor and lab-on-a-chip technologies to biomarker detection in biological fluids. Austin J Nanomed Nanotechnol 2 (1): 1009.
Ref10: Real-time measurement of small molecules directly in awake, ambulatory animals. Arroyo-Curras N, Somerson J, Vieira P A, Ploense K L, Kippin T E, Plaxco K W. Proc Natl Acad Sci USA. 2017 Jan. 24; 114 (4): 645-650
Ref11: Magnetic quantitative reverse transcription PCR: A high-throughput method for mRNA extraction and quantitative reverse transcription PCR Ricarda Jost, Oliver Berkowitz, and Josette Masle The Australian National University, Canberra, Australia BioTechniques 43:206-211 (August 2007) doi 10.2144/000112534

What is claimed is:

1. A blood analysis system comprising:
a centrifugation unit to receive blood of a subject, the centrifugation unit is to:
hold first capturing molecules, wherein one first capturing molecule binds with, to chemically capture, one molecule or ion that causes coagulation of the plasma, wherein the binding of one first capturing molecule with a molecule or an ion renders the bound molecule or ion unfunctional; and
centrifuge to suspend cellular components with a minimal plasma along with a number of the first capturing molecules;
a detection unit to receive the plasma with remaining number of the first capturing molecules and unbound molecules and ions, but without the cellular components, wherein the detection unit is to detect, and determine concentration of, one or more types of molecules and/or ions in the received plasma; and
a correction unit, coupled to the detection unit, the correction unit comprising:
a first cell to receive, from the detection unit, the plasma and having a first semi-permeable membrane (SPM) of a specific molecular weight cut-off that at least allows the plasma with the one or more types of molecules and/or ions to pass through; and
a second cell to receive, from the first detection unit, the plasma and having a second SPM, the second SPM being the same as the first SPM,
wherein one of the first cell or the second cell is operated to correct the determined concentration of the one or more types of molecules and/or ions, and
wherein the other of the first cell or the second cell is operated for declogging.

2. The blood analysis system of claim 1, wherein, in response to determining the concentration of the one or more types of molecules and/or ions being more than permissible concentration, the first cell or the second cell, being operated to correct the determined concentration, is operated to:
receive, in a chamber on a retentate side of the first or second SPM, second capturing molecules having concentration depending on an excess amount of the one or more types of molecules and/or ions that are to be captured and removed from the plasma for correcting the determined concentration, wherein the second capturing molecules have molecular weight more than the specific molecular weight cut-off of the first and second SPM and bind with the one or more types of molecules and/or ions to make each bound molecule or ion unfunctional;
remove the first capturing molecules and the second capturing molecules from the chamber on the retentate side; and
cause the one or more types of molecules and/or ions, which are not bound to the second capturing molecules, along with other molecules and/or ions to pass through the first or second SPM to a filtrate side of the chamber.

3. The blood analysis system of claim 1, wherein, in response to determining the concentration of the one or more types of molecules and/or ions being less than permissible concentration, the first cell or the second cell, being operated to correct the determined concentration, is operated to:
receive, in a chamber on a retentate side of the first or second SPM, deficient amount of the one or more types of molecules and/or ions;
remove the first capturing molecules from the chamber on the retentate side; and
cause the one or more types of molecules and/or ions along with other molecules and/or ions to pass through the first or second SPM to a filtrate side of the chamber.

4. The blood analysis system of claim 1, wherein the blood analysis system further comprises a detection unit, in a channel coupled to chambers of filtrate sides of the first and second SPMs of the first and second cells, to detect, and determine concentration of, the one or more types of molecules and/or ions in the plasma output from the correction unit, wherein, in response to determining the concentration, in the plasma output from the correction unit, not equal to permissible concentration of the one or more types of molecules and/or ions, the blood analysis system is operated to flow the plasma back to the correction unit, wherein the correction unit is further operated to recorrect the concentration of the one or more types of molecules and/or ions.

5. The blood analysis system of claim 4, wherein, in response to determining the concentration, in the plasma output from the correction unit, substantially equal to the permissible concentration, the blood analysis system is operated to flow the plasma to the subject.

6. The blood analysis system of claim 1, wherein chambers on a retentate side of the first and second SPMs of each of the first cell and the second cell are coupled to a respective concentration unit and a respective detection unit to concentrate and determine, respectively, concentration of un-captured retentate molecules and/or ions during declogging of the first cell or the second cell and to correct the concentration of the one or more types of molecules and/or ions, based on the concentration of the un-captured retentate molecules and/or ions by the other of the first cell or the second cell.

7. The blood analysis system of claim 1, wherein the first SPM and/or the second SPM filter the one or more types of molecules and/or ions with or without at least one of centrifugation or external electromagnetic field.

8. The blood analysis system of claim 1, wherein the blood analysis system further comprises:
a detection unit to receive the plasma with the remaining number of the first capturing molecules and the unbound molecules and ions, but without the cellular components, wherein the detection unit is to detect, and determine concentration of, cations and/or anions in the received plasma; and
an ion separation unit coupled to the detection unit to receive the plasma from the detection unit, wherein the ion separation unit is to filter ions from the received plasma based on SPMs with or without at least one of centrifugation or external electromagnetic field, wherein the ion separation unit comprises:
a third cell and having a third SPM of a specific molecular weight cut-off that allows one of anions or cations to pass through; and
a fourth cell and having a fourth SPM, the fourth SPM being the same as the third SPM,
wherein one of the third cell or the fourth cell is operated to filter one of the anions or cations, and
wherein the other of the third cell or the fourth cell is operated for declogging.

9. The blood analysis system of claim 8, wherein the ion separation unit comprises an anion separation unit and a cation separation unit, and wherein the ion separation unit is to:
separate the anions before the cations; or
separate the cations before the anions.

10. The blood analysis system of claim 8, wherein the third SPM is positively charged and the fourth SPM are negatively charged, when the anion are separated before the cations, and wherein the third SPM is negatively charged and the fourth SPM are positively charged, when the cations are separated before the anions.

11. The blood analysis system of claim 8, wherein the blood analysis system further comprises:
a detection unit coupled to chambers on filtrate sides of the third SPM of the third cell and the fourth SPM of the fourth cell to detect, and determine concentration of, the filtered anions and cations; and
a correction unit coupled to the detection unit to correct the concentration of filtered anions and/or cations expelled from the detection unit.

12. The blood analysis system of claim 11, wherein the blood analysis system further comprises a detection unit to receive the corrected anions and/or cations and to detect, and determine concentration of, the corrected anions and/or cations output from the correction unit,
wherein, in response to determining the concentration of the corrected cations and/or anions being not equal to a permissible concentration thereof, the blood analysis system is operated to flow the cations and/or the anions the back to the correction unit, wherein the correction unit is further operated to recorrect the concentration of the cations and/or the anions, and
wherein, in response to determining the concentration of the corrected cations and/or anions being equal to the permissible concentration thereof, the blood analysis system is operated to flow the cations and/or the anions to the subject.

13. The blood analysis system of claim 8, wherein the blood analysis system further comprises:
a detection unit to receive ion-free plasma with the first capturing molecules expelled from the ion separation unit, wherein the detection unit is to determine concentration of one or more specific types of molecules in the received plasma; and
one or more filtration units coupled to the detection unit to receive ion-free plasma with the first capturing molecules expelled from the ion separation unit, wherein one filtration unit is to filter a specific type of molecules based on SPMs with or without at least one of centrifugation or external electromagnetic field, from the ion-free plasma, wherein each of the one or more filtration units comprises:
a fifth cell and having a fifth SPM of a specific molecular weight cut-off that allows molecules with molecular weight equal to or less than the specific molecular weight cut-off to pass through; and
a sixth cell and having a sixth SPM, the sixth SPM being the same as the fifth SPM,
wherein one of the fifth cell or the sixth cell is operated to filter the specific type of molecules, and
wherein the other of the fifth cell or the sixth cell is operated for declogging.

14. The blood analysis system of claim 13, wherein the blood analysis system further comprises:
a detection unit coupled to chambers on retentate sides of the fifth SPM of the fifth cell and the sixth SPM of the sixth cell to detect, and determine concentration of, the filtered specific type of molecules expelled from the retentate side of the fifth cell or the sixth cell; and
a correction unit coupled to the detection unit to correct the concentration of filtered specific type of molecules expelled from the detection unit.

15. The blood analysis system of claim 14, wherein the blood analysis system further comprises a detection unit to receive the corrected specific type of molecules and to detect, and determine concentration of, the corrected specific type of molecules output from the correction unit,
wherein, in response to determining the concentration of the corrected specific type of molecules being not equal to a permissible concentration thereof, the blood analysis system is operated to flow the specific type of molecules the back to the correction unit, wherein the correction unit is further operated to recorrect the concentration of the specific type of molecules, and
wherein, in response to determining the concentration of the corrected specific type of molecules being equal to the permissible concentration thereof, the blood analysis system is operated to flow the specific type of molecules to the subject.

16. The blood analysis system of claim 13, wherein the molecules from filtration sides of the fifth SPM of the fifth cell and the sixth SPM of the sixth cell are returned to the subject or passed to a next filtration unit of the blood analysis system.

17. The blood analysis system of claim 1, wherein the blood analysis system further comprises:
one or more filtration units coupled to the detection unit to receive the plasma with remaining number of the first capturing molecules and unbound molecules and ions, but without the cellular components, wherein one filtration unit is to filter a specific type of molecules based on SPMs with or without at least one of centrifugation or external electromagnetic field, from the received plasma, wherein each of the one or more filtration units comprises:
a fifth cell and having a fifth SPM of a specific molecular weight cut-off that allows molecules with molecular weight equal to or less than the specific molecular weight cut-off to pass through; and
a sixth cell and having a sixth SPM, the sixth SPM being the same as the fifth SPM,
wherein one of the fifth cell or the sixth cell is operated to filter the specific type of molecules, and
wherein the other of the fifth cell or the sixth cell is operated for declogging.

18. The blood analysis system of claim 17, wherein the blood analysis system further comprises:
a detection unit coupled to chambers on retentate sides of the fifth SPM of the fifth cell and the sixth SPM of the sixth cell to detect, and determine concentration of, the filtered specific type of molecules expelled from the retentate side of the fifth cell or the sixth cell; and
a correction unit coupled to the detection unit to correct the concentration of filtered specific type of molecules expelled from the detection unit.

19. The blood analysis system of claim 18, wherein the blood analysis system further comprises a detection unit to receive the corrected specific type of molecules and to detect, and determine concentration of, the corrected specific type of molecules output from the correction unit,
wherein, in response to determining the concentration of the corrected specific type of molecules being not equal to a permissible concentration thereof, the blood analysis system is operated to flow the specific type of molecules the back to the correction unit, wherein the correction unit is further operated to recorrect the concentration of the specific type of molecules, and
wherein, in response to determining the concentration of the corrected specific type of molecules being equal to the permissible concentration thereof, the blood analysis system is operated to flow the specific type of molecules to the subject.

20. The blood analysis system of claim 17, wherein the molecules from filtration sides of the fifth SPM of the fifth cell and the sixth SPM of the sixth cell are returned to the subject or passed to a next filtration unit of the blood analysis system.

21. The blood analysis system of claim 1, wherein the blood analysis system comprises a holding unit coupled to the centrifugation unit to receive the plasma with the remaining number of the first capturing molecules and unbound molecules and ions, but without the cellular components from the centrifugation unit, wherein the received plasma is diluted with water in the holding unit to control the viscosity of the received plasma.

22. The blood analysis system of claim 1, wherein the detection unit comprises a single channel and a spectroscopy-based measurement unit, an optical sensing unit, a non-chemical sensing unit, an electrochemical sensing unit, a chemical reaction-based measurement unit, or a combination thereof, installed in the single channel to determine the concentration of the one or more types of molecules and/or ions.

23. The blood analysis system of claim 1, wherein the detection unit comprises:
one or more pairs of bifurcation channels, wherein a pair of bifurcation channels is to bifurcate plasma from a previous bifurcation channel or from an inlet channel, through which plasma for detection, and determining concentration, of the one or more types of molecules and/or ions is received, into two substantially equal plasma streams;
a spectroscopy-based measurement unit, an optical sensing unit, a non-chemical sensing unit, electrochemical sensing unit, a chemical reaction-based measurement unit, or a combination thereof, installed in each bifurcation channel to determine a concentration of the one or more types of molecules and/or ions in the respective bifurcation channel; and
one or more converging channels, wherein each converging channel is to combine two plasma streams from a pair of bifurcation channels into a single plasma stream.

24. The blood analysis system of claim 4, wherein the blood analysis system further comprises a concentration unit to receive the plasma output from the correction unit and to provide the plasma to the detection unit, the concentration unit comprising:
a seventh cell with a symmetrical arrangement of SPMs and a channel to receive plasma in a region between the symmetrical arrangement of SPMs;
a eighth cell with a symmetrical arrangement of SPMs and a channel to receive plasma in a region between the symmetrical arrangement of SPMs; and
an arrangement to allow water to circulate through the seventh cell and the eight cell,
wherein one of the seventh cell and the eighth cell is operated to increase a concentration of the plasma and the other of the seventh cell and the eighth cell is operated for declogging,
wherein the water is circulated to create an osmotic pressure in the regions between the symmetrical arrangement of SPMs and confine concentrated plasma to an area of the seventh cell or an area of the eighth cell that is being operated for concentration of the plasma.

25. The blood analysis system of claim 1, wherein the blood analysis system comprises:
a correction unit coupled to the centrifugation unit to receive the minimal plasma having the first capturing molecules and the cellular components from the centrifugation unit, the correction unit is to extract the first capturing molecules from the minimal plasma.

26. The blood analysis system of claim 25, wherein the minimal plasma having the cellular components is infused back to the subject along with replaced captured molecules and/or ions, and wherein the extracted first capturing molecules are discarded.

27. The blood analysis system of claim 26, wherein the minimal plasma having the cellular components are suspended in an isotonic solution, and optionally with oxygen and glucose, before infusing back of the subject.

28. The blood analysis system of claim 1, wherein the blood analysis system comprises:
an SPM and/or a centrifuge to separate platelets from the minimal plasma;
a detection unit to receive the separate platelets and count the platelets; and
a correction unit to correct the count of the platelets before infusing the minimal plasma with the corrected platelets to the subject.

29. The blood analysis system of claim 28, wherein the minimal plasma, after separating the platelets, is ultra-centrifuged to separate granulocytes from the minimal plasma, and wherein the blood analysis system comprises:
a detection unit to count the granulocytes; and
a correction unit to correct the count of the granulocytes, before infusing the minimal plasma with the corrected granulocytes to the subject.

30. The blood analysis system of claim 28, wherein the minimal plasma, after separating the platelets, is ultra-centrifuged to separate red blood cells (RBCs) from the minimal plasma, and wherein the blood analysis system comprises:
a detection unit to count the RBCs; and
a correction unit to correct the count of the RBCs, before infusing the minimal plasma with the corrected RBCs to the subject.

31. The blood analysis system of claim 2, wherein molecules and/or ions, equivalent to the molecules and/or the ions captured by the removed first capturing molecules, are added before infusing the plasma back to the subject.

32. The blood analysis system of claim 1, wherein the blood analysis system comprises a control unit to cut-off or cause a flow of plasma and/or cellular component at each channel of the blood analysis system via flow rate pumps.
